(19) United States
(12) Patent Application Publication
(10) Pub. No.: US 2005/0026147 A1
(43) Pub. Date: Feb. 3, 2005

(54) Methods and compositions for amplification of dna

(76) Inventors: Christopher L. Walker, Glen Carbon, FL (US); Ernest J. Mueller, Kirkwood, MO (US); Kevin J. Kayser, Chesterfield, MO (US); Jason S. Milligan, O'Fallon, FL (US); Erik R. Eastlund, Fenton, MO (US)

Correspondence Address: THOMPSON COBURN, LLP, ONE US BANK PLAZA, SUITE 3500, ST LOUIS, MO, 63101, US

(21) Appl. No.: 10/469,725

(22) PCT Filed: Jul. 29, 2003
(86) PCT No.: PCT/US03/23782

(51) Int. Cl. C12Q 1/68; C12N 9/22; C12N 1/08

(52) U.S. Cl. 435/6; 435/199; 435/270

(57) ABSTRACT

The invention provides an Enzyme Blend comprising a DNA polymerase and a DNA repair enzyme. Methods and kits for amplification of DNA that is damaged, undamaged, or suspected of being damaged are also provided.

### BACKGROUND OF THE INVENTION

1. Field of Invention

The present invention relates to compositions and methods for amplification of deoxyribonucleic acids, damaged or not.

2. Description of Related Art

DNA carries the genetic information of all living cells. An organism's genetic and physical characteristics, its genotype and phenotype, respectively, are controlled by precise nucleic acid sequences in the organism's DNA. The genome contains the sum total of all of the sequence information present in an organism's DNA. The nucleic acid sequence of a DNA molecule consists of a linear polymer of four nucleotides. The four nucleotides, each consisting of: (1) one of the four heterocyclic bases, adenine (“A”), cytosine (“C”), guanine (“G”) and thymine (“T”); (2) the pentose sugar derivative 2-deoxyribose which is bonded by its 1-carbon atom to a ring nitrogen atom of the heterocyclic bases; and (3) a monophosphate monoester formed between a phosphoric acid molecule and the 5′-hydroxy group of the sugar moiety. The nucleotides polymerize by the formation of diesters between the 5′-phosphate of one nucleotide and the 3′-hydroxy group of another nucleotide to give a single strand of DNA. In nature, two of these single strands interact by hydrogen bonding between complementary nucleotides; A complementary with T, and C complementary with G, to form base-pairs which result in the formation of the DNA double helix described by Watson and Crick. RNA is similar to DNA except that the base thymine is replaced by uracil (“U”) and the pentose sugar is ribose itself rather than deoxyribose. In addition, RNA exists in nature predominantly as a single strand.

Cells reproduce by duplicating their contents and then dividing in two. In the chromosome cycle, through DNA replication, the nuclear DNA is duplicated. After DNA replication, the cells undergo mitosis, in which the duplicate copies of the genome are separated. Accordingly, absent some malfunction, each progeny cell carries the same genetic information as that of its parent cell.

Polymerase chain reaction (PCR) exploits certain characteristics of DNA replication. Basic PCR techniques are disclosed in U.S. Pat. Nos. 4,683,202, 4,683,195 and 4,800,159. In its simplest form, PCR is an in vitro method for the enzymatic synthesis of specific DNA sequences, using two oligonucleotide primers that hybridize to opposite singular strands and flank the specific sequence of interest in the target DNA.

DNA polymerase, such as Taq, uses single-stranded DNA as a template for the synthesis of a complementary new strand. Heating double-stranded DNA to temperatures near boiling separates the double strands into single-stranded DNA templates. The DNA synthesis begins with adding specifically designed oligonucleotide primers to the DNA template. The primers then anneal to the template. DNA polymerase initiates synthesis of a new strand of DNA starting from the primer and using the DNA strand as a template. Both DNA strands serve as templates for synthesis provided specific oligonucleotide primer is supplied for each strand. The reaction mixture is then heated to separate the original strand and newly synthesized strands, which are then available for further cycles of primer hybridization, DNA synthesis, and strand separation. A repetitive series of reaction steps involving template denaturation, primer annealing, and the extension of the annealed primers by DNA polymerase results in the exponential amplification of a specific fragment. The 5′ ends of the primers define the termini of the fragment. Thus, the desired portion of even a very small amount of a DNA sample can be amplified.

PCR does not require highly purified DNA, and DNA released from boiling or lysis of cells may be used directly without purification. PCR may also be used to study the pattern of gene expression: mRNA converts into a cDNA by reverse transcription, and the cDNA then serves as the template for the PCR. DNA sequences do not have to be isolated before amplification by PCR, because the oligonucleotide primers determine the specificity of the reaction. PCR can amplify a DNA sample from a variety of sources, such as blood serum, saliva, semen, viruses, cells (prokaryotic or eukaryotic), and tissue sections. Even ancient DNA from Egyptian mummies several thousand years old can be amplified by PCR. Mycobacterium tuberculosis complex DNAs from Egyptian mummies were characterized by spoligotyping. See e.g., Zink, A R. et al., J Clin Microbiol., 41(1):359-67 (January 2003).

Most PCR amplifications of DNA performed today use Taq polymerase. However, purified Taq DNA polymerase enzyme completely lacks 3′ to 5′ exonuclease activity and thus cannot excise mis-inserted nucleotides (Tindall, et al., Biochemistry, 29:5226-5231 (1990)). Several independent studies suggest that 3′ to 5′ exonuclease-dependent proofreading enhances the fidelity of DNA synthesis. Reyland et al., J. Biol. Chem., 263:6518-6524 (1988); Kunkel et al, J. Biol. Chem., 261:13610-13616 (1986); and Bernard et al., Cell, 58:219-228 (1989).

Polymerase induced mutations incurred during PCR increase exponentially as a function of cycle number. For example, if an average of two mutations occur during one cycle of amplification, 2 million mutations will be made and propagated after 10 cycles and 2 trillion will exist after 20 cycles. Each mutant and wild type template DNA molecule will be amplified exponentially during PCR and thus a large percentage of the resulting amplification products will contain mutations. As a general rule, PCR applications that require high fidelity DNA synthesis cannot be done with standard Taq polymerase due to problems with mutations during DNA amplification.

To solve this problem, researchers have isolated or modified existing DNA polymerases to improve fidelity and/or thermostability of the polymerases for PCR. For example, the improved DNA polymerase increases the 3′→5′ exonuclease or proofreading activity. (see e.g. U.S. Pat. No. 6,489,150). Alternatively, the DNA polymerase is mixed with a small amount of an enzyme that exhibits the 3′→5′ proofreading activity. Because PCR employs high temperatures, especially during the denaturation step, preferably, the polymerases should also be thermostable. Takara et al., U.S. Pat. No. 5,436,149, describes a polymerase with enhanced thermostability.

Improved DNA polymerases can adequately amplify undamaged DNA. The proofreading capability of the improved DNA polymerases helps repair mis-incorporation of nucleotides during PCR. However, the integrity of the initial DNA template is a major factor in the success of PCR amplification. Even before the initial PCR, the DNA template may be damaged from its original state (whether known or not) under certain conditions such as exposure to sunlight or suboptimal storage conditions. Sites in the damaged DNA block progression of DNA polymerases, resulting in a low or undetectable amount of PCR product. The proofreading capability in standard or improved DNA polymerases cannot adequately repair such damaged templates to restore PCR progression because the proofreading capability simply improves the accuracy of the final product.

DNA damage may occur through oxidation, deamination, alkylation, depurination, or depyrimidination. Even normal cellular metabolic processes may generate numerous mutagenic DNA base lesions. In nature, these damaged bases may block DNA polymerase progression and halt DNA replication in cells. As a basic survival mechanism, cells activate special DNA polymerases and DNA repair enzymes that can synthesize DNA past such blocking lesions. However, such translesion synthesis, by its very nature, is mutagenic because the identity of the inserted base cannot be derived without correct base-pairing interactions with template nucleotides.

Depending on the type of DNA damage, cells develop various DNA repair enzymes, such as O6-alkylguanine-DNA alkyltransferase, deoxyuridine triphosphate pyrophosphatases, glycosylases, and apurinic/apyrimidinic (AP) endonucleases.

Fromenty et al. described a method for PCR of long DNA by supplementing the PCR reaction mixture with a DNA repair enzyme. (Fromenty, B. et al., Nucleic Acid Res., 28(11):i-viii (2000); see also PCT/FR01/00057 or Publication No. WO 01/51656). AP endonucleases, such as exonuclease III, has been isolated and studied as a repair tool in PCR. Exonuclease III is also referred to as AP endonuclease VI (“Endo VI”). Shida, T. et al., J Nucleic Acids Res., 24(22):4572-6 (Nov. 15, 1996). Because of the 3′→5′ exonuclease activity of AP endonuclease VI, which removes mononucleotides from the recessed 3′-termini of the DNA, PCR amplification of small fragments of damaged DNA can be especially problematic due to the destruction of the DNA from the exonuclease activity.

Fromenty et al. attempted, but failed, to rescue mouse genomic DNA that was damaged by phenol-chloroform extraction. Fromenty et al. observed “decreased amplification” of stored “phenol-extracted mouse DNA samples that had yielded no 8636 bp mtDNA [mitochondrial DNA] PCR fragment and little 316 bp PCR product (i.e. DNA samples that were undoubtedly severely damaged) in preliminary PCR experiments.” Fromenty at vi. Given the obstacles that Fromenty et al. encountered and failed to overcome, Fromenty et al. proceeded to amply DNA fragments having sizes of no more than 15 kb. Even then, the amplified product had low yield and poor specificity. Moreover, Fromenty's results show that small fragments of DNA could be amplified by PCR with or without exonuclease III, (see FIG. 1, p. iii of Fromenty et al., supra), which suggest that the DNA were not damaged or that the exonuclease III played no role in the results. However, when the small fragments of mouse DNA was extracted with phenol-chloroform, which Fromenty theorized would severely damage the DNA, Fromenty's method failed to rescue the small fragments of DNA. (Fromenty stated at vi that “[t]aken together, these data suggest that exonuclease III is unable to restore amplification of short PCR fragments from extensively degraded DNA templates”). Thus, Fromenty taught away from the use of exonuclease III.

Qbiogene Molecular Biology introduced a kit, under the trademark Auroris™, containing undisclosed enzyme(s) for PCR amplification of damaged DNA. Two enzyme mixes were provided in the kit, one for “long” DNA (longer than 7 kb) and the other for “short” DNA (between 500 bp and 4 kb). The technical bulletin included with the kit claimed that the enzyme mixes lacked endonuclease activity.

Therefore, a need still exists for a convenient means to repair damaged DNA of various sizes and to amplify the repaired DNA. It is desirable to amplify the DNA with substantial fidelity, specificity, and high yields. Cloning and expression experiments, cDNA analysis and array work can be improved by using accurate and high-yield amplifications.

All references cited herein are hereby incorporated by reference in their entirety.

### SUMMARY OF THE INVENTION

Accordingly, the present invention provides compositions, methods for repair, amplification, and rescue of DNA that is damaged, undamaged, or suspected of being damaged. Further, the present invention provides compositions and improved methods for amplification of undamaged DNA.

In part, this invention relates to an Enzyme Blend comprising a DNA polymerase and a means for repairing an apurinic/apyrimidinic (AP) damage in DNA. Preferably, the means for repairing an AP damage in DNA is an AP endonuclease DNA repair enzyme. The DNA polymerase may be modified to have proofreading capability. Alternatively, the DNA polymerase may be mixed with an enzyme that has proofreading capability, and an aliquot of the mix is used in the preparation of the Enzyme Blend. The DNA polymerase and the AP endonuclease DNA repair enzyme are mixed together to form an Enzyme Blend that can be conveniently handled, stored, and used as a single enzymatic entity. Although the Enzyme Blend comprises at least two different enzymes, surprisingly, the Enzyme Blend may be conveniently stored at a common temperature and under common conditions. The same Enzyme Blend can be used to rescue DNA fragments of 50 bp to 22 kb in size. The quality and efficiency of the DNA polymerase in the Enzyme Blend affect the size of the DNA that the Enzyme Blend can rescue. For example, AccuTaq™ LA DNA Polymerase can amplify a DNA of up to 40 kb. The present inventors, therefore, contemplate that the Enzyme Blend will rescue DNA larger than 22 kb, for example up to around 40 kb. If a DNA polymerase that can amply a 100 kb DNA were used in the Enzyme Blend of the instant invention, the present inventors contemplate that the Enzyme Blend can rescue a DNA having a size of up to about 100 kb. In other words, the upper size limit of the DNA for amplification by the Enzyme Blend is that of the DNA polymerase used in the blend.

In another aspect, the invention provides for a kit comprising an Enzyme Blend that comprises a DNA polymerase and a means for repairing an AP damage in DNA. Preferably, the means for repairing an AP damage in DNA is an AP endonuclease DNA repair enzyme.

In another aspect, the invention provides a method for repairing DNA that is damaged or suspected of being damaged comprising: a) forming a mixture comprising the DNA, an effective amount of the Enzyme Blend of the present invention, and deoxynucleoside 5′ triphosphates; and b) incubating the mixture at 0° C.-99° C. from about 0 sec. to about 3 hrs, more preferably at 0-50° C. from about 0 to about 1 hr.

In another aspect, the invention provides a method for amplification of DNA that is damaged, undamaged, or suspected of being damaged comprising: a) forming a mixture comprising the DNA, an effective amount of the Enzyme Blend of the present invention, deoxynucleoside 5′ triphosphates, and a pair of oligonucleotide primers, wherein the pair of oligonucleotide primers is substantially complementary to segments of the DNA; b) preincubating the mixture at 0° C.-99° C. from about 0 sec. to about 3 hrs; c) denaturing the DNA; and d) amplifying the DNA.

In another aspect, the invention provides for a method for amplification of DNA that is damaged, undamaged, or suspected of being damaged comprising: a) forming a mixture comprising the DNA, an effective amount of the Enzyme Blend of the present invention, and deoxynucleoside 5′ triphosphates; b) preincubating the mixture at a temperature of 0° C.-99° C. from about 0 sec. to about 3 hrs.; c) denaturing the DNA; d) incubating the mixture at a temperature sufficient to inactivate an AP endonuclease DNA repair enzyme in the Enzyme Blend and for a duration of time necessary to add a pair of oligonucleotide primers to the mixture, wherein the pair of oligonucleotide primers is substantially complementary to segments of the DNA; e) adding the pair of oligonucleotide primers to the mixture; and f) amplifying the DNA.

In another aspect, the invention provides a method for preparation of an Enzyme Blend comprising combining a DNA polymerase with a means for repairing an AP damage in DNA in a vessel to form a blend. Preferably, the means for repairing an AP damage in DNA is an AP endonuclease DNA repair enzyme.

In a further aspect, the invention provides a method for amplification of DNA that is damaged, undamaged, or suspected of being damaged comprising: a) forming a mixture comprising the DNA, an effective amount of DNA polymerase, an effective amount of a means for repairing an AP damage in DNA, deoxynucleoside 5′ triphosphates, and a pair of oligonucleotide primers, wherein the pair of primers is substantially complementary to segments of the DNA; b) preincubating the mixture at 0° C.-99° C. from about 0 sec. to about 3 hrs.; c) denaturing the DNA; and d) amplifying the DNA, wherein the DNA has a size from about 50 base pairs to about 500 base pairs or has a size from about 15,500 base pairs to about 22,000 base pairs. Preferably, the means for repairing an AP damage in DNA is an AP endonuclease DNA repair enzyme.

In another aspect, the invention provides a method for rescue of a DNA that is damaged or suspected of being damaged comprising: a) forming a mixture comprising the DNA, an effective amount of DNA polymerase, an effective amount of a means for repairing an AP damage in DNA, and deoxynucleoside 5′ triphosphates; b) preincubating the mixture at a temperature of 0° C.-99° C. from about 0 sec. to about 3 hrs.; c) denaturing the DNA; d) incubating the mixture at a temperature sufficient to inactivate the AP endonuclease DNA repair enzyme and for a duration of time necessary to add a pair of oligonucleotide primers to the mixture, wherein the pair of primers is substantially complementary to segments of the DNA; e) adding the pair of oligonucleotide primers to the mixture; and f) amplifying the DNA, wherein the DNA has a size from about 50 base pairs to about 500 base pairs or has a size from about 15,500 base pairs to about 22,000 base pairs. Preferably, the means for repairing an AP damage in DNA is an AP endonuclease DNA repair enzyme.

In another aspect, the invention provides an improved method for amplification of undamaged DNA comprising: a) forming a mixture comprising the DNA, an effective amount of a DNA polymerase, deoxynucleoside 5′ triphosphates, and a pair of oligonucleotide primers having thiophosphate linkages, wherein the pair of primers is substantially complementary to segments of the DNA; b) denaturing the DNA; and c) amplifying the DNA.

### BRIEF DESCRIPTION OF THE FIGURES

FIG. 1 shows that the Enzyme Blend of the present invention rescues λ DNA, which were damaged with formic acid in two different time periods, 7.5 minutes and 10 minutes. Lanes 1 and 2 show the amplification of undamaged λ DNA. Lanes 3 and 12 show PCR markers, from bottom to top: 50, 150, 300, 500, 750, 1000, 1500 and 2000 base pairs. Lanes 4 and 5 show the amplification of a 742 bp fragment of DNA damaged for 7.5 minutes using standard Taq DNA polymerase. Lanes 6 and 7 show the amplification of DNA damaged for 7.5 minutes using the Enzyme Blend, where 25 units of AP endonuclease VI was used in the Enzyme Blend. Lanes 8 and 9 show amplification of DNA damaged for 10 minutes using standard Taq DNA polymerase. Lanes 10 and 11 show amplification of DNA damaged for 10 minutes using the Enzyme Blend, where 25 units of AP endonuclease VI were used.

FIG. 2 shows a comparison of an amplification of human genomic DNA (Skb amplicon (Beta Globin gene)(“hgDNA”)) using the Enzyme Blend and an amplification of the same DNA with a DNA polymerase. The amplification included a step for inactivation of the AP endonuclease DNA repair enzyme in the Enzyme Blend. The hgDNA was intentionally damaged by exposing the sample to increasing amounts of formic acid (creating abasic sites). Lane 1 shows a PCR marker, from bottom to top: 50, 100, 200, 300, 400, 500, 750, 1000, 2000, 3000, 4000, 6000, 8000, and 10,000 base pairs. Lanes 2 and 3 show negative controls. Lanes 4 and 5 show amplification of undamaged hgDNA (5 ng/μl) with DNA polymerase. Lanes 6-11 show amplification with DNA polymerase of damaged hgDNA that have been treated with formic acid for 3, 10, and 15 minutes, respectively. The reactions were performed and loaded side-by-side onto the gel in duplicates. The negative controls were run in lanes 12 and 13. Lanes 14 and 15 show amplification of original or undamaged hgDNA with the Enzyme Blend. Lanes 16-21 show amplification with the Enzyme Blend of damaged hgDNA that have been treated with formic acid for 3, 10, and 15 minutes, respectively. The reactions were performed in duplicates and loaded side-by-side onto the gel.

FIG. 3 shows a sequencing analysis of damaged DNA repaired with the Enzyme Blend of the instant invention. The results of the analysis confirmed that the Enzyme Blend rescued the damaged λ DNA. The top electrophoretogram shows a sequencing attempt on a damaged template. The lower electrophoretogram is the same template after rescue with the Enzyme Blend.

FIG. 4A shows amplification with DNA polymerase of degraded murine genomic DNA templates (“mgDNA” from wild type (“wt”) mouse pups #1, 2, 3, 6, and 7) after phenol/chloroform extraction. Lane 6 is a PCR 100 bp low ladder, from bottom to top: 100, 200, 300, 400, 500, 600, 700, 800, 900, 1000 bp. Lane 7 shows amplification of freshly isolated mgDNA samples (from wt mouse pup #24) with DNA polymerase after a preincubation step. The mgDNA samples were subject to a phenol/chloroform extraction. The top band is a 627 bp amplicon (Substance P) and the bottom band is a 289 bp amplicon (FABP—fatty acid binding protein).

FIG. 4B shows the rescue of mgDNA using the Enzyme Blend. A preincubation step was performed to repair the DNA. Lanes 1 and 2 show amplification of DNA from wt pup #3 with AccuTaq™ LA DNA polymerase (P) and the Enzyme Blend (EB), respectively. Lanes 3 and 4 show amplification of DNA from wt pup #6 with AccuTaq™ LA DNA polymerase and the Enzyme Blend, respectively. Lanes 5 and 6 show amplification of DNA from wt pup #7 with AccuTaq™ LA DNA polymerase and the Enzyme Blend, respectively. Lanes 7 and 12 show PCR 100 bp ladders. Lanes 8 and 9 show amplification of DNA from wt pup #23 with AccuTaq™ LA DNA polymerase and the Enzyme Blend, respectively. Lanes 10 and 11 show amplification of DNA from wt pup #24 with AccuTaq™ LA DNA polymerase and the Enzyme Blend, respectively.

FIG. 5 shows rescue of damaged (or degraded) murine genomic DNA templates (after phenol/chloroform extraction). Lane 1 shows the amplification of DNA from wt pup #1 with DNA polymerase (P), but without a preincubation step. Lanes 2, 3, and 4 show rescue of DNA from wt pup #1 with the Enzyme Blend and after a preincubation of the mixture on ice for 1, 3, and 5 minutes, respectively. Lanes 5, 10, and 15 show PCR 100 bp ladders. Lane 6 show amplification of DNA from a knockout (“K/O”) mouse pup #2 with DNA polymerase and without a preincubation step. Lanes 7, 8, and 9 show rescue of DNA from K/O pup #2 with the Enzyme Blend and after preincubation of the mixture on ice for 1, 3, and 5 minutes, respectively. Lane 11 shows amplification of DNA from wt pup #24 with a DNA polymerase and without a preincubation step. Lanes 12, 13 and 14 show rescue of DNA from wt pup #24 with the Enzyme Blend and after a preincubation on ice for 1, 3, and 5 minutes, respectively.

FIG. 6 shows that the Enzyme Blend can be stored at room temperature or 37° C. for at least a month. Lanes 1 and 2 show the amplification of original or undamaged λ DNA with the Enzyme Blend. Lanes 3 and 10 show the PCR markers, from bottom to top: 50, 150, 300, 500, 750, 1000, 1500 and 2000 bp. Lanes 4 and 5 show amplification of DNA damaged for 7.5 minutes with formic acid and treated with a DNA polymerase. Lanes 6 and 7 show rescue of DNA damaged for 7.5 minutes with formic acid and treated with the Enzyme Blend that has been stored at 37° C. for one month. Lanes 8 and 9 show rescue of DNA damaged for 7.5 minutes with formic acid and treated with the Enzyme Blend that has been stored at room temperature for one month.

FIG. 7 shows that the Enzyme Blend can be stored at room temperature for at least 6 weeks. Lanes 1 and 2 show the amplification of original or undamaged λ DNA with the Enzyme Blend. Lanes 3 and 10 show the PCR markers, from bottom to top: 50, 150, 300, 500, 750, 1000, 1500 and 2000 bp. Lanes 4 and 5 show amplification of DNA damaged for 7.5 minutes with formic acid using standard Taq DNA polymerase. Lanes 6 and 7 show rescue of DNA damaged for 7.5 minutes with formic acid using the Enzyme Blend that has been stored at room temperature for 6 weeks. The results shown in FIGS. 6 and 7 strongly support that the stability of the Enzyme Blend can be maintained for at least a year at −20° C.

FIG. 8 shows rescue of damaged human genomic DNA 294 bp amplicon using the Enzyme Blend. Lanes 1 and 8 show the PCR marker, from bottom to top: 50, 150, 300, 500, 750, 1000, 1500 and 2000 bp. Lanes 2 (undamaged), 4 (damaged for 10 min.), and 6 (damaged for 3 min.) show the rescue of the human genomic DNA with the Enzyme Blend (E.B.). Lanes 3 (undamaged), 5 (damaged for 10 min.), and 7 (damaged for 3 min.) show the amplification of the hgDNA with DNA polymerase (P).

FIG. 9 shows that the Enzyme Blend rescued damaged DNA templates of different concentrations. The DNA was damaged for 3 minutes or 7 minutes. Lanes 1, 10, and 19 show PCR markers, from bottom to top: 50, 150, 300, 500, 750, 1000, 1500, and 2000 base pairs. Lanes 2 and 3 show the amplification with standard DNA polymerase of genomic DNA (10 ng) after damage treatment for 3 minutes. Lanes 4 and 5 show rescue of a 3-minute damaged gDNA (10 ng) with the Enzyme Blend. Lanes 6 and 7 show the amplification of gDNA (10 ng) after damaged treatment of 7 minutes with standard DNA polymerase. Lanes 8 and 9 show the rescue of a 7-minute damaged gDNA (10 ng) with the Enzyme Blend. Lanes 11 and 12 show amplification of gDNA (100 ng) with standard DNA polymerase. The gDNA was treated for 3 minutes with formic acid to damage the DNA. Lanes 13 and 14 show the rescue of the 3-minute damaged gDNA (100 ng) with the Enzyme Blend. Lanes 15 and 16 show the amplification of gDNA (100 ng) with standard DNA polymerase. The gDNA was treated for 7 minutes with formic acid to damage the gDNA. Lanes 17 and 18 show rescue of a 7-minute damaged gDNA with the Enzyme Blend.

FIG. 10 shows that addition of thiophosphate nucleotides to the 3′ end of the primers can replace the use of a manual inactivation step or “hot-start” in the amplification of DNA (527 bp). Lanes 1 and 2 show amplification of undamaged hgDNA (200 ng/μl) with thiophosphate primers without a hotstart. Lane 3 shows PCR marker, from bottom to top: 50, 150, 300, 500, 750, 1000, 1500 and 2000 bp. Lanes 4 and 5 show amplification of undamaged hgDNA (200 ng/μl) with non-thiophosphate primers and a non-hotstart PCR. Lanes 6 and 7 show amplification of original or undamaged hgDNA using non-thiophosphate primers, but with a hot start step. The Enzyme Blend was used in each of the above amplifications.

FIG. 11 shows the rescue of heat and formic acid damaged hgDNA with an Enzyme Blend of a DNA polymerase and Endonuclease IV (“Endo IV”) with or without a preincubation step. For the top and bottom sections of FIG. 11, Lane 1 is a wide range PCR marker, from bottom to top: 50, 100, 200, 300, 400, 500, 750, 1000, 2000, 3000, 4000, 6000, 8000, and 10,000 base pairs. The first half of the top section of FIG. 11 shows amplification with DNA polymerase without Endo IV (top lanes 2 (undamaged, 50 ng), 3 (undamaged, 5 ng), 4 (damaged by heat at 99° C. for 1 min., 50 ng), 5 (damaged by heat at 99° C. for 1 min., 5 ng), 6 (damaged by heat at 99° C. for 3 min., 50 ng), 7 (damaged by heat at 99° C. for 3 min., 5 ng), 8 (damaged by formic acid for 1 min., 10 ng), 9 (damaged by formic acid for 1 min., 1 ng), 10 (damaged by formic acid for 5 min., 10 ng), and 11 (damaged by formic acid for 5 min., 1 ng)). Lane 12 shows a negative control.

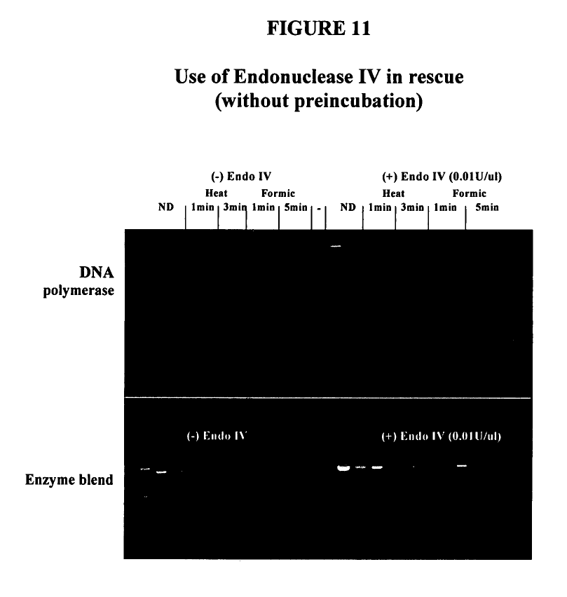

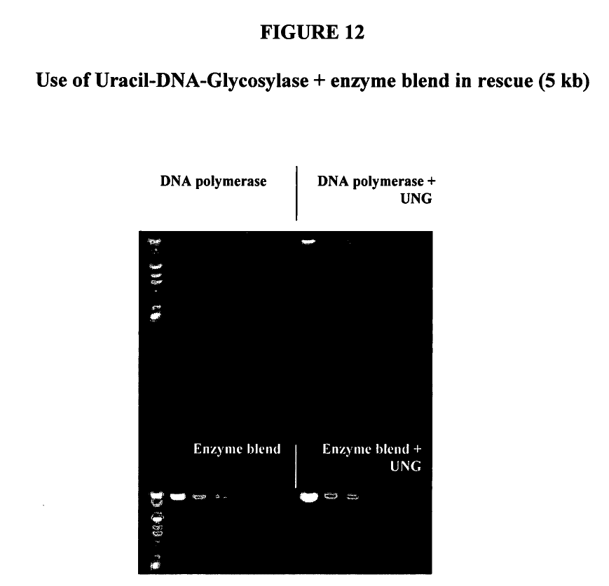

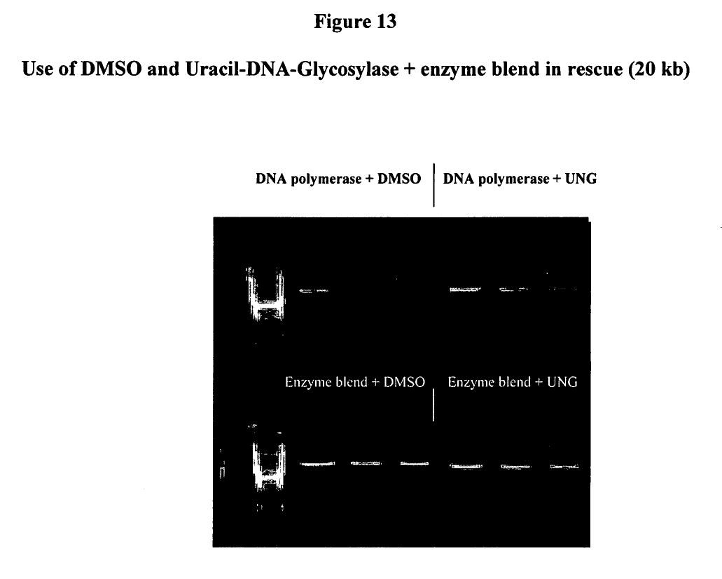

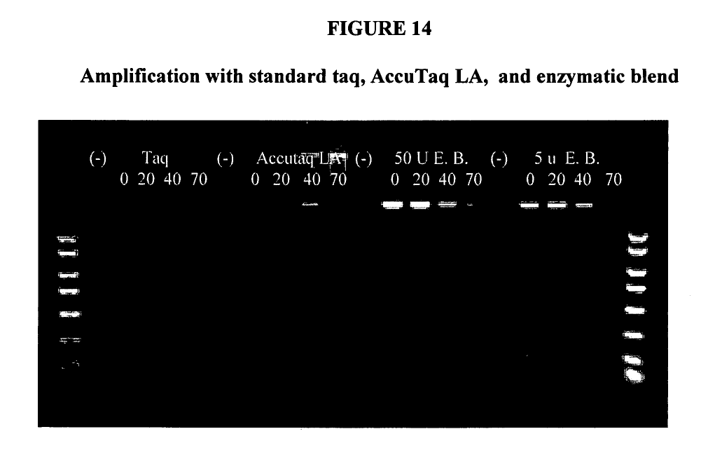

The second half of the top section of FIG. 11 shows amplification with DNA polymerase and Endo IV (top lanes 13 (undamaged, 50 ng), 14 (undamaged, 5 ng), 15 (damaged by heat at 99° C. for 1 min., 50 ng), 16 (damaged by heat at 99° C. for 1 min., 5 ng), 17 (damaged by heat at 99° C. for 3 min., 50 ng), 18 (damaged by heat at 99° C. for 3 min., 5 ng), 19 (damaged by formic acid for 1 min., 10 ng), 20 (damaged by formic acid for 1 min., 1 ng), 21 (damaged by formic acid for 5 min., 10 ng), and 22 (damaged by formic acid for 5 min., 1 ng)).

The first half of the bottom section of FIG. 11 shows the amplification and rescue of the DNA with the Enzyme Blend without Endo IV. The first half of the top section of FIG. 11 shows amplification with DNA polymerase without Endo IV (bottom lanes 2 (undamaged, 50 ng), 3 (undamaged, 5 ng), 4 (damaged by heat at 99° C. for 1 min., 50 ng), 5 (damaged by heat at 99° C. for 1 min., 5 ng), 6 (damaged by heat at 99° C. for 3 min., 50 ng), 7 (damaged by heat at 99° C. for 3 min., 5 ng), 8 (damaged by formic acid for 1 min., 10 ng), 9 (damaged by formic acid for 1 min., 1 ng), 10 (damaged by formic acid for 5 min., 10 ng), and 11 (damaged by formic acid for 5 min., 1 ng)). Lane 12 shows a negative control.

The second half of the bottom section of FIG. 11 shows amplification and rescue with the Enzyme Blend and Endo IV (bottom lanes 13 (undamaged, 50 ng), 14 (undamaged, 5 ng), 15 (damaged by heat at 99° C. for 1 min., 50 ng), 16 (damaged by heat at 99° C. for 1 min., 5 ng), 17 (damaged by heat at 99° C. for 3 min., 50 ng), 18 (damaged by heat at 99° C. for 3 min., 5 ng), 19 (damaged by formic acid for 1 min., 10 ng), 20 (damaged by formic acid for 1 min., 1 ng), 21 (damaged by formic acid for 5 min., 10 ng), and 22 (damaged by formic acid for 5 min., 1 ng)).

FIG. 12 shows rescue of damaged DNA, a 5 kb amplicon (Beta globin gene), using the Enzyme Blend with uracil DNA glycosylase (UNG). Lane 1 of the top and bottom sections of FIG. 12 shows a wide range marker. The first half of the top section of FIG. 12 shows amplification with DNA polymerase (top lanes 2 (undamaged, 5 ng, 50 U of Endo VI), 3 (undamaged, 5 ng, 5 U), 4 (damaged by heat at 99° C. for 1 min., 5 ng, 50 U), 5 (damaged by heat at 99° C. for 1 min., 5 ng, 5 U), 6 (damaged by heat at 99° C. for 3 min., 5 ng, 50 U), and 7 (damaged by heat at 99° C. for 3 min., 5 ng, 5 U)).

The second half of the top section of FIG. 12 shows amplification with DNA polymerase and UNG (top lanes 8 (undamaged, 5 ng, 50 U of Endo VI), 9 (undamaged, 5 ng, 5 U), 10 (damaged by heat at 99° C. for 1 min., 5 ng, 50 U), 11 (damaged by heat at 99° C. for 1 min., 5 ng, 5 U), 12 (damaged by heat at 99° C. for 3 min., 5 ng, 50 U), and 13 (damaged by heat at 99° C. for 3 min., 5 ng, 5 U)).

The first half of the bottom section of FIG. 12 shows the amplification and rescue with the Enzyme Blend (bottom lanes 2 (undamaged, 5 ng, 50 U of the Enzyme Blend), 3 (undamaged, 5 ng, 5 U), 4 (damaged by heat at 99° C. for 1 min., 5 ng, 50 U), 5 (damaged by heat at 99° C. for 1 min., 5 ng, 5 U), 6 (damaged by heat at 99° C. for 3 min., 5 ng, 50 U), and 7 (damaged by heat at 99° C. for 3 min., 5 ng, 5 U)).

The second half of the bottom section of FIG. 12 shows amplification with the Enzyme Blend and UNG (bottom lanes 8 (undamaged, 5 ng, 50 U of the Enzyme Blend), 9 (undamaged, 5 ng, 5 U), 10 (damaged by heat at 99° C. for 1 min., 50 ng, 50 U), 11 (damaged by heat at 99° C. for 1 min., 50 ng, 5 U), 12 (damaged by heat at 99° C. for 3 min., 5 ng, 50 U), and 13 (damaged by heat at 99° C. for 3 min., 50 ng, 5 U)).

FIG. 13 shows rescue of damaged hgDNA (heat damaged), a 20 kb amplicon (Beta globin gene), using DMSO and uracil DNA glycosylase (UNG). As depicted in lanes 1-6 of the bottom section of FIG. 13, the absence of smearing in the wells indicates that the rescue with the Enzyme Blend results in products with greater specificity, especially for large amplicons, than the products obtained by amplification with standard DNA polymerase. Lane 1 shows the λ Hind III marker, from bottom to top; 125, 564, 2027, 2322, 4361, 6557, 9416, and 23130 bp. The top section of FIG. 13 shows amplification with DNA polymerase and DMSO or UNG. Lanes 2, 3, and 4 show amplification of 200 ng, 100 ng, and 50 ng respectively, of undamaged hgDNA with DNA polymerase and DMSO. Lanes 5, 6 and 7 show amplification of 200 ng, 100 ng, and 50 ng of undamaged hgDNA with DNA polymerase and UNG. The bottom section of FIG. 13 shows amplification with the Enzyme Blend and DMSO or UNG. Lane 1 is the λ Hind III marker. Lanes 2, 3, and 4 show is 200 ng, 100 ng, and 50 ng of original or undamaged hgDNA with the Enzyme Blend and DMSO. Lanes 5, 6, and 7 show amplification of 200 ng, 100 ng, and 50 ng of undamaged hgDNA with the Enzyme Blend and UNG.

FIG. 14 shows a comparison of the amplification of depurinated hgDNA with standard Taq DNA polymerase (Taq), AccuTaq™ LA DNA polymerase (AccuTaq™ LA), and the Enzyme Blend (“E.B.”). Lanes 1 and 22 show the PCR markers. Lanes 2, 7, 12, and 17 are negative controls. Lanes 3, 8, 13, and 18 show amplification of hgDNA that was depurinated for 0 min. with Taq, AccuTaq™ LA, 50 U of the Enzyme Blend, and 5 U of the Enzyme Blend, respectively. Lanes 2, 7, 12, and 17 are negative controls. Lanes 4, 9, 14, and 19 show amplification of hgDNA that was depurinated for 20 min. with Taq, AccuTaq™ LA, 50 U of the Enzyme Blend, and 5 U of the Enzyme Blend, respectively. Lanes 5, 10, 15, and 20 show amplification of hgDNA that was depurinated for 40 min. with Taq, AccuTaq™ LA, 50 U of the Enzyme Blend, and 5 U of the Enzyme Blend, respectively. Lanes 6, 11, 16, and 21 show amplification of hgDNA that was depurinated for 70 min. with Taq, AccuTaq™ LA, 50 U of the Enzyme Blend, and 5 U of the Enzyme Blend, respectively.

### DETAILED DESCRIPTION OF THE INVENTION

Definitions.

“Amplification” refers to the duplication of a DNA template. Non-limiting examples of amplification methods include the polymerase chain reaction and the rolling circle amplification. For a discussion of rolling circle amplification, see Lizardi P M et al., Nat Genet.,19(3):225-32 (July 1998), which is incorporated by reference in its entirety.

“Complementary” refers to the base pairing of the nucleotide bases, G to C and A to T, through hydrogen bonds between the oligonucleotide primer and the DNA template. Perfect (100%) complementation is not required for amplification of the DNA because amplification conditions can be adjusted to accommodate mismatching or wobbling between the bases in the primer and DNA template. The oligonucleotide primer may be “substantially complementary” to the DNA template, such that the extension product synthesized from one primer, when separated from its complement, can serve as a template for the extension product of the other primer. A person of ordinary skill in the art will appreciate that some oligonucleotide primer may contain degenerate nucleotides. A non-limiting example of a degenerate nucleotide is inosine. The oligonucleotide primer is at least 40% complementary, preferably at least 70% complementary, more preferably at least 80% complementary, and still more preferably at least 90% complementary to the DNA template.

“Damaged DNA” means DNA that has been damaged (altered) or suspected of being damaged from its original, unaltered (pristine) state, whether known or not. Such damage may be caused by, but is not limited to, deamination, depurination, depyrimidination, oxidation, alkylation, and UV irradiation. One way DNA may be suspected of being damaged is when, for either known or unknown reasons, it cannot be amplified or can only be poorly amplified using standard or modified DNA polymerases. For example, if the DNA has been intentionally or unintentionally aged or improperly stored (e.g. in 4° C. or −20° C. refrigeration), and/or an amplification reaction of the DNA results in either low or undetectable amount of products on a gel, such DNA could be suspected of being damaged. Non-limiting examples of sources from which DNA can be obtained include animal tissues (e.g blood, saliva, hair, skin, etc.), cells and cell lysates, viruses, isolated DNA, bacteria, plant tissue, and environmental samples (e.g. sediment, soil, water). The source may be prokaryotic or eukaryotic. The DNA need not be purified. However, if purification is desired, non-limiting purification techniques include, inter alia, phenol-chloroform extraction, gradient centrifugation (e.g CsCl), and ion exchange chromatography.

Although sequencing is a common and easy method to determine and quantify, if desired, the damage to a DNA, other additional methods exist to quantify DNA damage. For example, the Kubo method (Dojindo) examines oxidative damage. Oxidative damage to DNA is a result of the interaction of DNA with reactive oxygen species (ROS), in particular, the hydroxy radical which is converted from a superoxide and a hydrogen peroxide by the Fenton reaction. Hydroxy radicals produce a multiplicity of modifications in DNA. Oxidative attack by hydroxy radical on the deoxyribose moiety will lead to the release of free bases from DNA, generating strand breaks with various sugar modifications and simple AP sites. They are a major type of damage generated by ROS. It has been estimated that endogeneous ROS can result in about 2×10⁵ base lesions per cell per day.

The Kubo method uses an Aldehyde Reactive Probe (ARP) reagent (N′-aminooxymethylcarbonylhydrazino-D-biotin). ARP reacts specifically with an aldehyde group, which is the open ring form of the AP sites. This reaction makes it possible to detect DNA modifications that result in the formation of an aldehyde group. After treating DNA containing AP sites with the ARP reagent, the AP sites are tagged with biotin residues. By using an excess amount of ARP, all AP sites can be converted to biotin-tagged AP sites. Therefore, AP sites can be quantified using avidin-biotin assay followed by a colorimetric detection of peroxidase or alkaline phosphatase conjugated to the avidin (see e.g. Lindahl & Nyberg, 1972; Kubo et al., 1992, which is incorporated by reference in its entirety).

Another method for determining DNA damage is the (Trevigen) CometAssay, which provides rapid analysis of DNA fragmentation associated with DNA damage. The comet assay or single cell gel electrophoresis assay is based on the alkaline lysis of labile DNA at sites of damage. The unwound, relaxed DNA is able to migrate out of the cell during electrophoresis and can be visualized by SYBR Green® staining. Cells that have accumulated DNA damage appear as fluorescent comets with tails of DNA fragmentation or unwinding, whereas, normal undamaged DNA does not migrate far from the origin. For further reading of the determination of DNA damage, please see e.g. Kim B S et al., “New measure of DNA repair in the single-cell gel electrophoresis (comet) assay,” Dept. of Applied Statistics, Yonsei University, Seoul, South Korea, Environ Mol Mutagen., 40(1):50-6 (2002); Chakrabarti S. et al., “Fluorescent labelling of closely-spaced aldehydes induced in DNA by bleomycin-Fe(III),” Int J Radiat Biol., 75(8):1055-65 (Aug. 1999); Loureiro, Ana Paula M. et al., “Development of an On-Line Liquid Chromatography-Electrospray Tandem Mass Spectrometry Assay to Quantitatively Determine 1,N2-Etheno-2′-deoxyguanosine in DNA,” Chem. Res. Toxicol., 15:1302-1308 (2002); Pinto M et al., “Quantification of DNA damage by PFGE: development of an analytical approach to correct for the background distribution,” Int J Radiat Biol. Jun; 76(6): 741-8 (June 2000); Basnakian A G and James S J., “Quantification of 3′ OH DNA breaks by random oligonucleotide-primed synthesis (ROPS) assay,” DNA Cell Biol, 1996 March;15 (3): 255-62 (March 1996). The above references are incorporated by reference in their entirety.

“Undamaged DNA” is DNA that can be amplified using standard or modified DNA polymerase. The DNA is not necessarily 100% in its original, unaltered (pristine) state. Theoretically, most, if not all, DNA, especially long DNA, have some amount of damage. However, the amount or extent of the damage is insignificant or insufficient to halt or impair the progression of the DNA polymerase during DNA synthesis. As discussed below, in comparison to standard or modified DNA polymerase, the Enzyme Blend can improve the amplification of undamaged DNA.

An “AP endonuclease DNA repair enzyme” means an enzyme that repairs DNA damage at apurinic sites.

“Enzyme Blend” means a blend of at least a DNA polymerase and an AP endonuclease DNA repair enzyme, and which is used, handled, and stored as a single enzymatic entity. Such Enzyme Blends may, but need not, be included as components in a kit.

A person of ordinary skill in the art appreciates that in any amplification reaction, the amount of enzyme(s), primers, template, and other components in the reaction may be adjusted as necessary to obtain a desired result. An “effective amount” of the Enzyme Blend refers to the amount that is necessary to achieve the desired level of amplification of a DNA. For example, about 0.05 to 3 units of the Enzyme Blend may be used to amplify a DNA of 20 bp to 22,000 bp.

“Repair” refers to the incorporation of one or more base pairs at one or more altered or damaged sites in the DNA using the Enzyme Blend to allow for amplification or increased amplification of the DNA as compared to undetectable or low amplification of the same DNA with standard or mixed DNA polymerases. By way of example and not a limitation, repair of the DNA can be confirmed by a number of means, e.g. by running the DNA sample on an electrophoresis gel or by sequencing the DNA sample, wherein superior results are generated on the repaired DNA sample as opposed to the sample treated with polymerase.

“Rescue” refers to the repair or substantial repair of damaged DNA and subsequent amplification of the repaired DNA. The rescue results in an increased yield and/or specificity of the DNA as compared to the low or undetectable yield of the DNA by amplification with standard or modified DNA polymerase.

“Room temperature” is scientifically defined as about 25° C. at 1 atmosphere. However, depending on a variety of environmental factors or conditions, such as pressure and/or humidity, the room temperature may fluctuate and still be within the scope of the present invention.

“Stabilizing agent” refers to a compound or solution that assists in maintaining the activity of the Enzyme Blend over a period of time. Non-limiting examples include glycerol, EDTA, 1,4-dithioerythritol, DL-dithiothreitol (DTT), 2-mercaptoethanol, 2-mercaptoethanolamine, fericyanide, hydrazine, borane, or phosphine.

“Thermostable” or “thermostability” means an enzyme is stable to heat and preferentially is active at higher temperatures, especially the high temperatures used for denaturation of DNA. More particularly, thermostable enzymes are not substantially inactivated at the temperatures used in polymerase chain reactions.

A “unit” of enzyme depends on the particular enzyme. Some non-limiting examples: a unit of DNA repair enzyme, e.g. Endonuclease VI, is defined as an amount of enzyme required to produce 1 nmol of mononucleotide in 30 minutes at 37° C. from sonicated DNA. A unit of DNA polymerase, e.g. AccuTaq™ LA DNA polymerase, is defined as an amount of enzyme required to catalyze the incorporation of about 10 nanomoles of deoxyribonucleotides into a polynucleotide in 30 minutes at 74° C. In a preferred Enzyme Blend, a unit of the Enzyme Blend refers to about 2.5 U of AccuTaq™ LA DNA polymerase and 50 U of Endonuclease VI.

The Enzyme Blend of the present invention allows amplification of damaged DNA samples that are otherwise an unsuitable template for conventional polymerases. The Enzyme Blend comprises a DNA polymerase and a means for repairing an AP damage in DNA., Preferably, the means for repairing an AP damage in DNA is an AP endonuclease DNA repair enzyme. The individual enzymes have optimal activities at different pH levels (AP endonuclease VI buffer is pH 7.0 and AccuTaq™ is pH 9.3). The present invention seeks to utilize conditions that maximize the combined activities of the separate enzymes.

In addition, the inventors determined conditions such that both enzymes can be stabilized for long term storage in a single blend at the same pH level. The common optimal pH for the blend of both enzymes depends on the specific DNA polymerase and AP endonuclease DNA repair enzyme used in the Enzyme Blend. For example, for the Enzyme Blend comprising AccuTaq™ LA DNA polymerase and Endonuclease VI, the functional pH range for both enzymes is about 7.5 to about 9.5, preferably, the optimal pH is about 9.3.

Moreover, the art teaches that the exonuclease activity of most Class II AP endonuclease degrades DNA. Accordingly, practitioners in the art normally do not put the DNA polymerase and AP endonuclease together in a blend and use the blend in an amplification reaction, such as standard PCR, because the AP endonuclease degrades the template DNA and/or primers. Through experimentation, the present inventors surprisingly discovered that an Enzyme Blend could be made and used to rescue damaged DNA. The present inventors discovered techniques, described in more detail below, that overcome the obstacles imposed by the AP endonuclease. For example, the mixture may be incubated on wet ice (˜0° C.), the reaction may be carried out using modified primers, such as phosphthioate primers, and/or the AP endonuclease, such as AP endonuclease VI, may be thermally inactivated prior to the addition of primers to the mixture.

Preferably, the DNA polymerase is AccuTaq™ LA DNA polymerase. AccuTaq™ LA DNA polymerase is an optimized blend of high quality Taq DNA polymerase and a small amount of an additional polymerase that exhibits 3′ to 5′ exonuclease or proofreading activity. Takara Shuzo Co., Ltd. owns U.S. Pat. Nos. 5,436,149 and 6,410,277 claiming the formulation of a DNA polymerase and certain additional DNA polymerases. These U.S. patents are incorporated by reference in their entirety. The AccuTaq™ LA DNA polymerase can be obtained from Sigma Aldrich, Catalog Number D8045. The proofreading capability of AccuTaq™ LA DNA polymerase may correct for mis-incorporation of nucleotides, which allows production of PCR products that are longer and more accurate. For example, AccuTaq™ LA DNA polymerase can increase the fidelity of the amplification of DNA by up to 6.5 times that of standard Taq DNA polymerase. AccuTaq™ LA DNA polymerase can efficiently and accurately produce products of up to about 22 kb on genomic templates and up to about 40 kb on less complex templates such as lambda or bacterial DNA.

Other DNA polymerase besides AccuTaq™ LA DNA polymerase may be used. Often, the DNA polymerase is stored in the presence of a non-ionic detergent, such as Tween-20. Preferably, the DNA polymerase is thermostable. Still more preferably, the DNA polymerase has equal to or better fidelity than AccuTaq™ DNA polymerase. Non-limiting examples include vent, deep vent, pwo, Taq, Tth, and Klentaq DNA polymerase. For damaged DNA with deaminated cytosines, preferably, the DNA polymerase does not bind to uracil.

Preferably, the AP endonuclease DNA repair enzyme is AP endonuclease VI, REF 1, APEX, Endonuclease IV, APNI, APE1 (human endonuclease 1), or FEN-1. More preferably, the DNA repair enzyme is AP endonuclease VI, which is also known as exonuclease III. AP endonuclease VI and exonuclease III are used interchangeably throughout this specification, including the figures and claims. AP endonuclease VI is a class II AP endonuclease that possesses 3′→5′ exonuclease, AP endonuclease, 3′ phosphomonoesterase, 3′-repair diesterase and RNAse H activities. Class II AP endonucleases cleave the DNA phosphodiester backbone 5′ of AP sites (apurinic/apyrimidinic sites) creating a free 3′-OH end for DNA polymerase elongation in PCR. AP endonuclease VI cleaves DNA-RNA hybrids using its intrinsic RNAse H activity. It degrades double-stranded DNA with a blunt end, resulting in a 5′ overhang or a nick.

The gap is filled in the 5′→3′ direction by DNA polymerase's nick translation activity. AP endonuclease VI incises DNA at AP sites via a Mg++ hydrolytic reaction mechanism. AP endonuclease VI removes the 5′ mononucleotides from the double-stranded DNA, leaving a free 3′ hydroxyl end, which can serve as a primer for DNA synthesis. Alternatively, AP endonuclease VI can degrade 3′-protruding ends of four bases or more in length and single-stranded DNA.

AP endonucleases generally have only minimal 3′→5′ exonuclease activity. Mol, C. D. et al., Mutat. Res., 460:211-29 (2000). An exception is AP endonuclease VI which has substantial 3′→5′ exonuclease activity. DNA repair is hypothesized to occur by AP endonuclease activity cleaving 5′ to an abasic site or damaged base creating a 3′ hydroxyl for DNA polymerase extension. The other hypothesized activities of AP endonucleases (3′-phosphomonoesterase, RNase H, 3′-repair diesterase), including the 3′→5′ exonuclease activity, may also play a role in DNA repair.

AP endonuclease 3′→5′ exonuclease activity is different from the 3′→5′ exonuclease proofreading activity of DNA polymerases. The 3′→5′ exonuclease proofreading activity of some thermostable DNA polymerases occurs at elevated temperatures during PCR cycling resulting in the removal of misincorporated deoxynucleotides without the continued 3′→5′ stepwise removal of mononucleotides. The 3′ 5′ exonuclease activity of AP endonucleases generally occurs at optimal temperatures for survival of the organism from which the AP endonucleases derived. (A thermostable AP endonuclease with 3′→5′ exonuclease activity would be active at elevated temperatures during PCR cycling). Substrates for this 3′→5′ exonuclease activity of AP endonuclease VI include blunt ends, 3′ termini and nicks in duplex DNA. The 3′→5′ exonuclease activity of AP endonucleases catalyzes the stepwise removal of mononucleotides from 3′-hydroxyl termini of duplex DNA mentioned previously. This stepwise removal of mononucleotides generally occurs at mesophilic temperatures and is not dependant on a DNA polymerase misincorporation of a deoxynucleotide.

There are two Class II AP endonuclease families based on the mechanism of cleavage. Members of either family may be used. Human APE1 class II AP endonuclease belongs to one family while Endo IV from E. coli belongs to the other family. Homologs of either family along with any enzyme that cuts 5′ to an AP site may also be used. An example of an enzyme cutting 5′ to an AP site as well as having DNA glycosylase activity is fpg offered by New England Biolabs.

In yeast, when an AP class II endonuclease cuts 5′ to an abasic site, a 5′ abasic moiety flap occurs by DNA polymerase synthesis displacement. FEN-1 is a structure specific endonuclease that cuts this flap in yeast. Preferably, the structure specific endonuclease is thermostable.

The Enzyme Blend may further comprise a stabilizing agent. The stability of the AP endonuclease VI in the Enzyme Blend is dependent in part on the oxidation state of the active site thiol. Maintaining this thiol in a reduced state can be accomplished by adding any number of stabilizing agents. The stabilizing agent may be 1,4-dithioerythritol, DL-dithiothreitol (DTT), 2-mercaptoethanol, 2-mercaptoethanolamine, fericyanide, hydrazine, borane, or phosphine. Preferably, the stabilizing agent is DTT. DTT is a reducing agent.

The Enzyme Blend may further comprise a ligase. Damaged double stranded DNA with unmodified 3′ hydroxyl and/or 5′ phosphorous termini can be covalently linked by ligase. A non-limiting example is T4 DNA ligase.

The Enzyme Blend may further comprise a photolyase. Photolyase catalyzes the repair of UV-light-induced DNA lesions. A non-limiting example is Thermus thermophilus photolyase.

The Enzyme Blend may further comprise a DNA glycosylase. Preferably, the DNA glycosylase is thermostable. Damaged DNA or ancient DNA that has cytosine deaminated to uracil cannot be restored by current methods. Damaged bases in the starting template can inhibit PCR or cause transitions in the sequence recovered after PCR. Deamination of cytosine to uracil causes a C→T change, resulting in a G→A transitions in the recovered PCR product. This mutation can be permanent and magnified with each round of amplification. To overcome this challenge, DNA glycosylase is added to the Enzyme Blend or to the reaction mixture. DNA glycosylases excise the incorrect bases. DNA glycosylases generate abasic sites that are more susceptible to subsequent DNA strand breaks by β-elimination. These strand breaks are 3′ of the abasic site and are not suitable for 3′ PCR elongation. Blocked 3′ termini can then be processed by a 5′ AP endonuclease. Preferably, the DNA glycosylase is a uracil N-glycosylase, which removes uracil bases. More preferably, the DNA glycosylase is used with an Enzyme Blend comprising an AP endonuclease and a DNA polymerase that has 3′→5′ proofreading ability without binding tightly to uracil. See e.g. Greagg, M. et al., Proc. Natl. Acad. Sci. USA, 9045-9050 (1999), which is incorporated by reference in its entirety.

In an embodiment, the Enzyme Blend further comprises endonuclease IV or DMSO.

In another embodiment, the Enzyme Blend further comprises ligase, glycosylase, endonuclease, photolyase, and/or DMSO.

The Enzyme Blend allows amplification of DNA that is damaged by a number of means. Non-limiting examples include damage by acid, heat, oxidation, or reaction with organic chemicals. These processes create damages in the DNA that mimic the damages believed to be sustained by DNA in nature. The Enzyme Blend is designed to rescue any environmentally damaged DNA sample, including samples from non-living tissues. Non-limiting examples of non-living tissues are fossilized plant or animal remains, mummified organisms, DNA that has been isolated or purified and aged, or tissue sample or whole blood that have aged. The Enzyme Blend can also amplify commercially purchased genomic DNA preparations or undamaged templates with higher yield than that obtained using DNA polymerase alone.

The Enzyme Blend may be present in a composition that is suitable for storage of the enzyme until its intended use, i.e. it exists as a storage stable composition. Storage stable compositions will typically comprise the Enzyme Blend in combination with a buffer medium. Buffer mediums of interest typically comprise: buffering agents, e.g. Tris-HCl, Tricine, HEPES, phosphate, etc.; solvents, e.g. water, glycerol, etc.; salts, e.g. KCl, NaCl, (NH₄)₂ SO₄, etc.; reducing agents, e.g. β-mercaptoethanol, DTT, DTE, etc.; chelating agents, e.g. EDTA, CDTA, etc.; detergents, e.g Triton X100; Tween 20, Thesit, NP40, etc.; proteins, e.g. BSA; and the like.

In a preferred embodiment, the Enzyme Blend comprises: a) 1-2.5 units/ul DNA polymerase; b) 5-50 units/ul AP endonuclease VI; c) 3-15 mM DTT; and d) 16-50% v/v glycerol.

More preferably, the Enzyme Blend comprises: a) 2.5 units/ul DNA polymerase; b) 5-50 units/ul AP endonuclease VI; c) 10 mM Tris-HCl pH 8.0; d) 150 mM KCl; e) 100 ug/ml BSA; f) 0.075 mM EDTA; g) 7.5 mM DTT; and h) 0.25% v/v Tween 20; I) 0.25% v/v IGEPAL CA-630; and j) 50% v/v glycerol.

The Enzyme Blend may be packaged in a kit, although it is not required. The kit may contain other components. For example, if the kit is for PCR, non-limiting examples of other components may include enzyme buffer(s), deoxynucleoside 5′ triphosphates (“dNTPs”), and/or salts.

The Enzyme Blend may be used to repair damaged DNA. There may be situations where the state of the DNA is unknown, but that due to the condition of the sample or because of other known facts, it is strongly suspected that the DNA has been damaged. For example, polymerase chain reaction using standard or modified DNA polymerase cannot amplify the DNA. In such cases, the Enzyme Blend would be useful to repair the suspected damage. Accordingly, the present invention provides a method for repairing DNA that is damaged or suspected of being damaged comprising: a) forming a mixture comprising the DNA, an effective amount of the Enzyme Blend, and deoxynucleoside 5′ triphosphates; and b) incubating the mixture at 0° C.-99° C. from about 0 sec. to about 3 hrs. The duration of the incubation time depends on the extent of the damage. Lightly damaged or undamaged DNA requires shorter amounts of incubation time to repair the templates, whereas heavily damaged DNA requires longer incubation times as a general rule. In some instances, the DNA may be so damaged that the Enzyme Blend cannot rescue the DNA. For example, the Enzyme Blend cannot rescue DNA that has been heated at high temperature (e.g. 99° C.) for a long period of time (depending on the size of the DNA).

The temperature of the incubation depends on the half-life of the AP endonuclease DNA repair enzyme used in the Enzyme Blend. For example, AP endonuclease VI optimally functions at 0-50° C. Thus, when AP endonuclease VI is used in the Enzyme Blend, the reaction may be incubated at 0-50° C. AP endonuclease VI's optimal catalytic activity is near 45° C., and exhibits diminished activity at a lower temperature. The AP endonuclease VI is thermally inactivated at temperature ≧50° C. (Hoheisel J D, Anal Biochem., 209(2):238-46 (March, 1993), which is incorporated by reference in its entirety). AP endonuclease VI may have residual activity at a high temperature, and thus, a preferred incubation of about at least 70° C. for approximately 1-30 minutes is used to inactivate it (New England Biolabs, Exonuclease III, Technical Bulletin and Product Insert). At high temperatures, the preincubation time used to sufficiently repair the damaged DNA by the Enzyme Blend for amplification of the DNA may be for a minute or less. Because the AP endonuclease VI has lower catalytic activity at 0° C., the incubation may be longer to sufficiently repair the DNA for amplification.

Range of temperature repair for AP endonuclease VI:

[image]

Additional information on the thermostability and catalytic activity of various enzymes, such as DNA repair enzymes, are readily available in the literature. See e.g., Adv Biochem Eng Biotechnol., 45:57-98 (1992). For example, E. coli endonuclease IV has functional activity from about 25° C. to about 70° C., while Thermotoga maritima endonuclease IV has functional activity from about 25° C. to about 90° C. See e.g. Haas, Brian J. et al., J. of Bacteriology, 181(9):2834-2839 (1999). Endonuclease III from E. coli has functional activity at about 37° C. Endonuclease III from archeon Pyrobaculum aerophilum has functional activity at about 70° C. See also, Coolbear, T. et al., Adv. Biochem. Eng. Biotechnol., 45:57-98 (1992). The above references are incorporated by reference in their entirety.

The present invention also provides for a method for amplification of DNA that is damaged, undamaged, or suspected of being damaged comprising: a) forming a mixture comprising the DNA, an effective amount of the Enzyme Blend, deoxynucleoside 5′ triphosphates, and a pair of oligonucleotide primers, wherein the pair of primers is substantially complementary to segments of the DNA sample; b) preincubating the mixture at 0° C.-99° C. from about 0 sec. to about 3 hrs.; c) denaturing the DNA; and d) amplifying the DNA. In one embodiment, the amplification is by PCR. In another embodiment, the amplification is by rolling circle amplification.

Although polymerase chain reaction (PCR) is the most popular DNA amplification method, there are other unique methods to amplify DNA. In another embodiment, the amplification is by rolling circle amplification. In rolling circle amplification, a non-thermostable polymerase, such as mesophilic Phi29 DNA polymerase (Current Opinion in Biotechnology, 13:65-67 (2002), which is incorporated by reference in its entirety) or Pol III from E. coli may be used. Rolling circle amplification (RCA) and strand displacement are forms of isothermal amplification. RCA is an isothermal process (constant temperature) that replicates using a DNA polymerase and oligonucleotide probes for amplification. Circular DNA and target probes together with mesophilic DNA polymerases are used to amplify thousands of copies of the single stranded circle. The isothermal strand displacement process uses a strand-displacing DNA polymerase. Primers hybridize to the displaced strands and the DNA polymerase extends the primers. Duplex DNA molecules are formed and then more single-stranded copies of the target are created.

A person of ordinary skill in the art will appreciate that the amplification condition may be adjusted as necessary, depending on a number of factors, such as the size of the DNA to be amplified and the primers used in the amplification reaction. A longer DNA requires shorter denaturation times. For example, a DNA having a size of less than about 3-4 kb may have a denaturation time between 30-60 seconds. A DNA having a size of about 5-8 kb may have a denaturation time of 20 seconds. Still further, a DNA having a size of greater than about 10 kb may have a denaturation time of 10 seconds. In PCR, the annealing temperature depends on the melting temperature of the primers used. Typically, elongation times should not exceed a minute for each kilobase of DNA. The amount of dNTPs to use may also be adjusted. For example, DNA having a size of less than about 4 kb require about 200 μM dNTPs. The amount of dNTP may be increased up to about 300 μM in a PCR amplification of DNA having a size longer than about 10 kb. In addition, more or fewer PCR cycles may be employed. Preferably, about 20 to about 60 cycles are used.

Oligonucleotide primers are usually 21 to 34 bases in size, but any other size may be used. The primers are designed to have a GC content of about at least 50%. Preferably, the melting temperatures (Tₘ) of the forward and reverse primers should be within 3° C. of each other and between 60° C. and 72° C. Primers should have minimal internal base-pairing sequences (i.e., potential hairpins) or any significant length of complementary regions between the two PCR primers. Primers may also be designed with a final CC, GG, CG, or GC on the 3′ end of the primers in order to increase priming efficiency.

The template may include nicked, damaged or undamaged DNA, or DNA suspected of being damaged. To repair the DNA, the DNA is incubated with the Enzyme Blend. The duration of the incubation time vary depending on the degree of damage in the DNA. Preferably, the incubation time is from about 0 second to about 3 hours at about 0° C. to about 99° C.

A DNA polymerase cofactor refers to a nonprotein compound on which the enzyme depends for activity. A number of such materials are known in the art, including manganese and magnesium compounds which release divalent manganese or magnesium ions in the aqueous reaction mixture. Useful cofactors include, but are not limited to, manganese and magnesium salts, such as chlorides, sulfates, acetates and fatty acid salts. The smaller salts, such as chlorides, sulfates and acetates, are preferred with magnesium chlorides and sulfates being most preferred. The magnesium concentration in the PCR may be varied. Preferably, the magnesium concentrations are about between 1 and 5 mM.

It will be appreciated that nonstandard PCR can be employed in the present invention. Examples include “touchdown” PCR, and secondary or “nested” PCR. For more discussion on other PCR techniques, see e.g. PCR 2, A Practical Approach Edited by M. J. McPherson, B. D. Hames and G. R. Taylor 1995 Oxford University Press; PCR Protocols: A Guide to Methods and Applications, M. A. Innis, D. H. Gelfand, J. J. Sninsky and T. J. White, Academic Press, 1990, 482 pp.; PCR Technology, Current Innovations, H. G. Griffin and A. M. Griffin, CRC Press, Boca Raton, Fla., 1994, 400 pp. The above references are incorporated by reference in their entirety.

The method of the present invention can amplify a DNA (that is damaged, undamaged, or suspected of being damaged) having a size of at least about 200 base pairs. The method may also amplify the DNA having a size of less than about 22,000 base pairs. Preferably, the method amplifies the DNA having a size of at least about 500 base pairs. Still more preferably, the method amplifies the DNA having a size of less than about 1,000 base pairs. More preferably, the method amplifies the DNA having a size of about 50 base pair to about 500 base pairs. Still more preferably, the method amplifies the DNA having a size of about 15,500 base pair to about 22,000 base pairs.

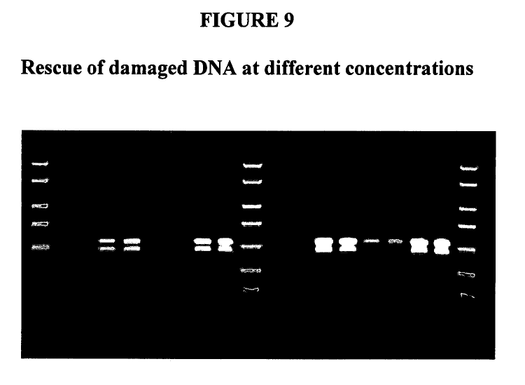

Unlike the method of Fromenty et al., the present inventions can amplify small DNA (DNA having sizes approximately at or below 500 base pairs) (see e.g. FIG. 9) either by using the Enzyme Blend or by stepwise additions of the AP endonuclease and DNA polymerase to the reaction mixture. Fromenty et al. failed to amplify small DNA for a number of possible reasons: 1) when they damaged the DNA (using heat at 99° C.) past the point where standard PCR could still amplify the template DNA, the DNA was too severely damaged to allow rescue even with exonuclease III (or Endonuclease VI); 2) they failed to damage the DNA sufficiently to observe recovery (using heat at 99° C.); 3) they failed to discover the right concentration of exonuclease III; and/or 4) exonuclease III might have degraded the primers in the PCR. Instead, they used a low concentration of exonuclease III in an attempt to avoid the highly active 3′→5′ exonuclease activity of the exonuclease III. In the present invention, the DNA was damaged by formic acid, which severely damaged the DNA. To rescue the damaged DNA, a high concentration of exonuclease III was added to the DNA. From time to time, the highly active 3′→5′ exonuclease activity degraded the primers and the small DNA template. To overcome this obstacle, the present inventors surprisingly discovered that the 3′→5′ exonuclease activity can be sufficiently impaired to prevent degradation of primers and templates by using primers with thiophosphate linkages or by a heat inactivation. Alternatively, the appropriate amount of the exonuclease III was used to achieve repair and thus, to rescue the small DNA, without degrading the primers and/or template. For exonuclease III (or AP endonuclease VI), the amount was generally about 5-50 units per microliter of the reaction volume.

As discussed above, occasionally, the AP endonuclease DNA repair enzyme may degrade the primers such that it impairs or reduces the efficiency of the amplification. On these occasions, the practitioner may use a pair of oligonucleotide primers that have thiophosphate linkages. Preferably, the thiophosphate linkages are located on the last two nucleotides at the 3′ end of each oligonucleotide primer. Alternatively, the practitioner may include an inactivation step that comprises incubating the mixture at a constant temperature (after an initial denaturation step) sufficient to inactivate the DNA repair enzyme and for a time sufficient to add the primers to the mixture. Typically, the time is less than a minute. Preferably, the constant temperature is about at least 70° C.

The present inventors contemplate another embodiment of the present invention in which amplification of undamaged DNA with DNA polymerase and oligonucleotide primers having thiophospate linkages will result in an increased yield of the DNA products as compared to amplification with DNA polymerase with primers that do not have thiophosphate linkages.

The present invention also provides a method for amplification of DNA that is damaged, undamaged, or suspected of being damaged comprising: a) forming a mixture comprising the DNA, an effective amount of the Enzyme Blend of the present invention and deoxynucleoside 5′ triphosphates; b) preincubating the mixture at a temperature of 0° C.-99° C. from about 0 sec. to about 3 hrs; c) performing a first incubation of the mixture at a temperature and for a duration of time sufficient to denature the DNA, preferably at about 94° C. for about 5 seconds; d) performing a second incubation of the mixture at a temperature, preferably of about 75° C., for a duration of time as needed to add a pair of oligonucleotide primers to the mixture, wherein the pair of primers is complementary to predetermined segments of the DNA sample; e) adding the pair of oligonucleotide primers to the mixture; and f) amplifying the DNA.

A combination of oligonucleotide primers with thiophosphate linkages and an inactivation step may be used in the reaction.

Any or all of the steps of the methods of the present invention may be automated. For high throughput application, a person of ordinary skill in the art will appreciate that commercially available robotics may be employed. For example, for PCR, a Perkin-Elmer DNA Cycler 9700 may be used in conjunction with a Biomek robot.

The Enzyme Blend of the present invention allows repair followed by amplification of ancient DNA samples. Ancient DNA may contain abasic sites, alkylated bases, single stranded nicks, areas of denaturation, or thymidine dimers. DNA extracted from nonliving tissue is typically of low average molecular weight and has undergone both hydrolytic and oxidative damages. Hydrolytic damage results in deamination of bases and in depurination and depyrimidation. Oxidized DNA bases found in ancient DNA have been shown to inhibit PCR. This oxidative damage may be caused directly by ionizing radiation or by free radicals. Single-stranded DNA breaks with nicks, gaps or protruding ends along with interstrand crosslinks occur frequently in ancient DNA samples. The repair and amplification of ancient DNA can improve with the addition of uracil-N-glycosylase to the Enzyme Blend.

The Enzyme Blend can be used to improve the amplification of undamaged DNA. As demonstrated in the examples below and in the figures (e.g FIGS. 13 and 14), use of the Enzyme Blend increased the quality, specificity, and yield of the DNA products as compared to the use of DNA polymerase alone.

The present invention has numerous applications. This invention can benefit any technique that relies on amplification of DNA, especially DNA that has been damaged or is suspected of being damaged. These techniques may be sequencing or restriction analysis of the amplified samples. Aged DNA samples are frequently damaged. Thus, other downstream applications include forensic identification (e.g. STR, AFLC, and microsatellite), organism typing, diagnostic identification of viral or bacterial diseases and analysis of suspected genetically modified organisms. Additionally, the present invention impacts other techniques that rely on amplification to generate sufficient amounts of DNA for later manipulation such as cloning from damaged DNA samples or generation of DNA libraries.

### EXAMPLES

The following table provides examples of sources to acquire the enzymes to make and use the Enzyme Blend:

| Component | Source | Address |
|---|---|---|
| Exonuclease III or AP | Trevigen | 8405 Helgerman Court |
| Endonuclease VI | | Gaithersburg, MD |
| | | 20877 |
| Exonuclease III or AP | TAKARA | Fisher Scientific |
| Endonuclease VI | | 2000 Park Lane Drive |
| | | Pittsburgh, PA |
| | | 15275-1126 |
| Exonuclease III or AP | New England Biolabs, | 32 Tozer Road |
| Endonuclease VI | Inc. | Beverly, MA |
| | | 01915-5599 |
| Fpg (fapy)-DNA | New England Biolabs, | |
| Glycosylase | Inc. | |
| Uracil DNA Glycosylase | New England Biolabs, | |
| | Inc. | |
| Uracil DNA Glycosylase | Invitrogen | Invitrogen |
| | | Life Technologies |
| | | 1600 Farady Avenue |
| | | Carlsbad, CA |
| | | 92008 |
| Heat Labile Uracil DNA | Epicentre | Epicentre |
| Glycosylase | | 726 Post Road |
| | | Madison, WI |
| | | 53713 |
| Endonuclease IV | Fermentas, Inc. | 7520 Connelly Drive, |
| | | Unit A, Hanover, MD |
| | | 21076 |
| APEI | Sigma-Aldrich | 3050 Spruce Street |
| | Corporation | St. Louis, MO 63103 |

### Example 1

Amplification Procedure.

The following example shows a general procedure for rescue of damaged DNA with the Enzyme Blend. The procedure may be adjusted as needed to achieve the desired result. For example, the concentration of the Enzyme Blend, template DNA, primers, and MgCl₂ may be adjusted, depending on the system being utilized. The following standard reagents were added to a thin-walled 200-μl or 500-μl heat-stable reaction vessel:

| | | Final |
|---|---|---|
| Volume | Reagent | Concentration |
| 5 μl | 10X Buffer for | 1X |
| | AccuTaq LA | |
| | DNA | |
| | Polymerase | |
| 1 μl | dNTP Mix (10 mM | 200 μM |
| | each) | |
| 1 μl | Template | 5-6 ng/μl |
| | DNA* | |
| | (5-6 ng/ul) | |
| 40 μl | Water | — |
| 1 μl | Enzyme | 2.5 units/μl |
| | Blend ™ | |
| 48 μl | Total Volume | |

The mixture was mixed gently and briefly centrifuged to collect all components to the bottom of the vessel. The reaction was subject to the following standard reaction conditions:

| Preincubation | 37° C. | 30-60 min |
|---|---|---|
| Initial denaturation | 94° C. | 5 sec |
| For an inactivation | 75° C. | Until |
| step | | restart |
| | Pause at 75° C., | |
| | add forward and | |
| | reverse primers, | |
| | then resume | |
| | program | |
| For cycles 1-30 | | |
| Denaturation | 94° C. | 5 sec |
| Annealing | Tm-5° C. | 20 sec |
| Extension | 68° C. | 1 min |
| | | per kb |
| Final extension | 68° C. | 1 min |
| | | per kb |

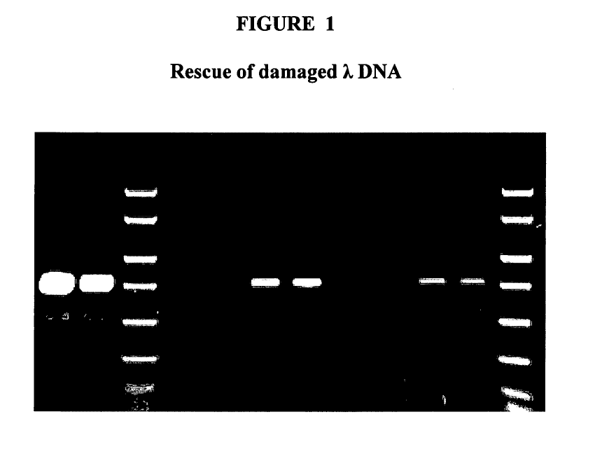

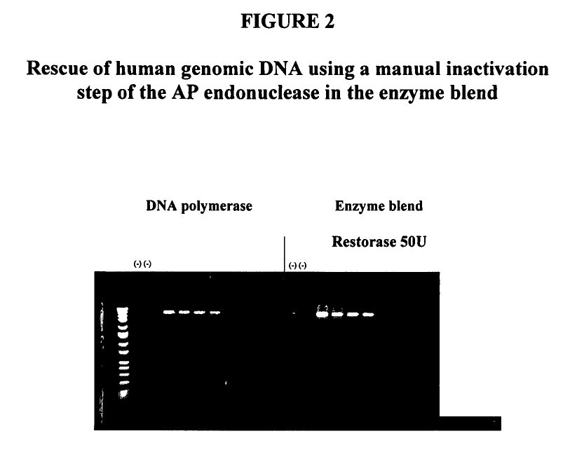

Each oligonucleotide primer was between 21 bases (high G+C content) and 34 bases (high A+T content) in length. Melting temperatures of the primers were around 62° C.-70° C. This was determined using the algorithm based upon nearest neighbor analysis of Rychlik and Rhoads, Nucl. Acids Res. 17:8543-8551 (1989), which is incorporated by reference in its entirety. The amplified DNA was evaluated using agarose gel electrophoresis and subsequent ethidium bromide staining (see Molecular Cloning: A Laboratory Manual, Third Edition, Sambrook, J., et al., (Cold Spring Harbor Laboratory Press, New York, 2000)). See FIG. 1 for an example of results that can be obtained by this amplification method.

### Example 2

This example demonstrates a sample preparation of the Enzyme Blend of the present invention. About 0.5 ul (2.5 units) of AccuTaq™ LA DNA polymerase, about 0.075 ul of 100 mM DTT, and about 0.5 ul (50 units) of AP endonuclease VI were added into a vessel and mixed together. About 1 ul of the resulting Enzyme Blend was used for each 50 ul total volume of the mixture for amplification. A scale-up preparation of the Enzyme Blend can be readily made and aliquoted into individual vessels. If the Enzyme Blend is used within two days, DTT is not used in the Enzyme Blend.

### Example 3

A DNA sample can derive from a number of different sources (cells, tissues, etc.) and may have been damaged by a number of different ways (age, chemical exposure, light exposure, etc.). This example demonstrates that the Enzyme Blend rescued intentionally damaged DNA. The DNA sample was damaged by formic acid to recreate apurinic/apyrimidinic damage typically observed in DNA damaged by natural processes.

Lambda DNA was intentionally damaged by exposure to formic acid. The bottom of a spin column was broken off and placed in a disposable tube. The tube was centrifuged for 2 minutes @ 3,000 RPM to form column. The tube was discarded. A mixture of 4 μl of ? DNA (2.5 ng/μl) was added to 20 μl 1× Tris-EDTA buffer and 10 μl of a 10× formic acid dilution (1 μl 96% Formic acid+10 μl H₂O). The mixture was incubated at 37° C. for 10 min. After placement in the column, the mixture was centrifuged for 4 min. at 3,000 RPM. A microliter of Tris-EDTA buffer (100×, 0.2 μM filtered) was added to stop the reaction.

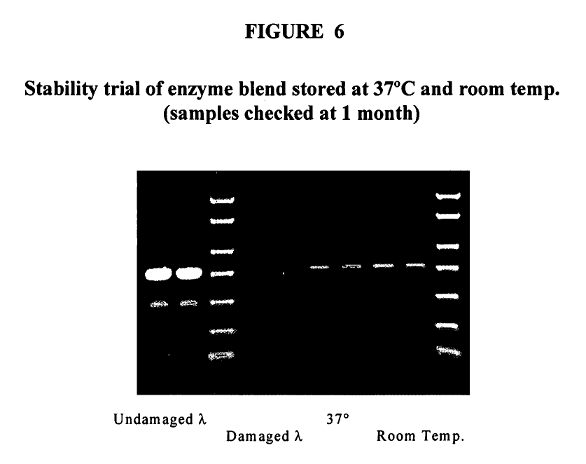

A mixture was prepared and amplified as described in Example 1. Examples of results can be seen in FIGS. 6, 7, and 11. These results show the successful rescue with the Enzyme Blend of DNA damaged by formic acid or heat. DNA polymerase could not amplify the damaged DNA (i.e. no bands of DNA were observed on the agarose gel). In contrast, the Enzyme Blend successfully rescued the damage DNA (i.e. strong bands of DNA were observed on the agarose gel).

### Example 4

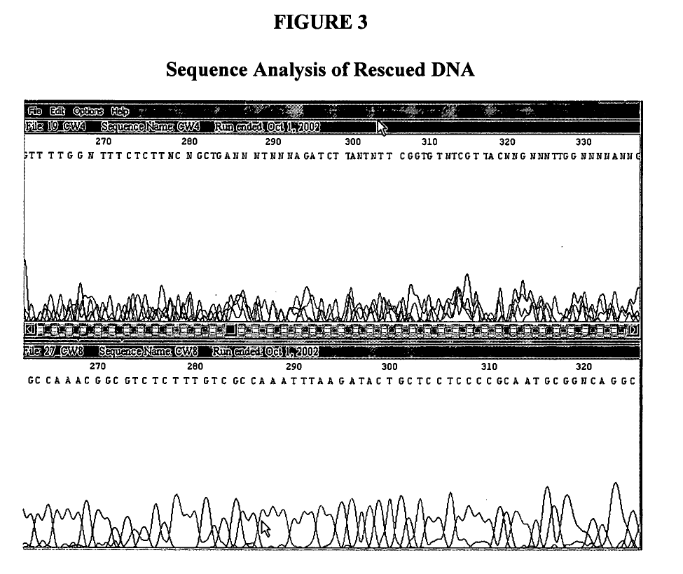

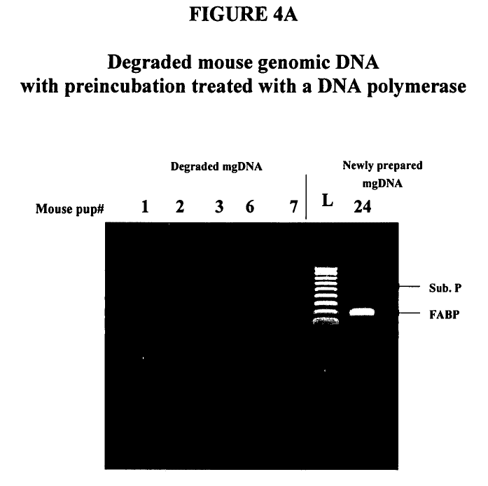

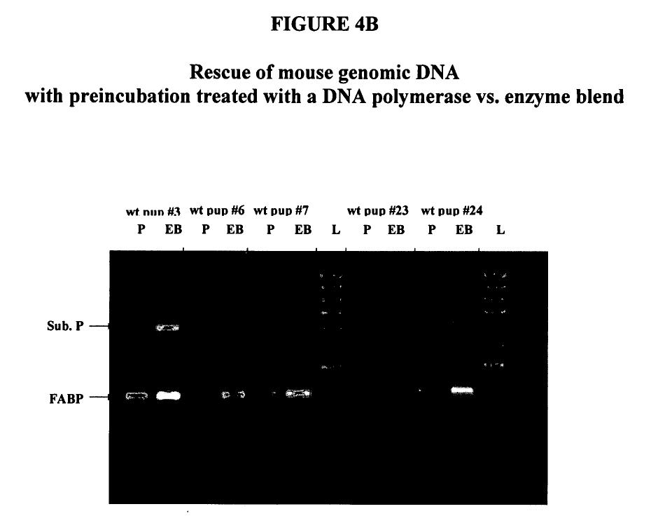

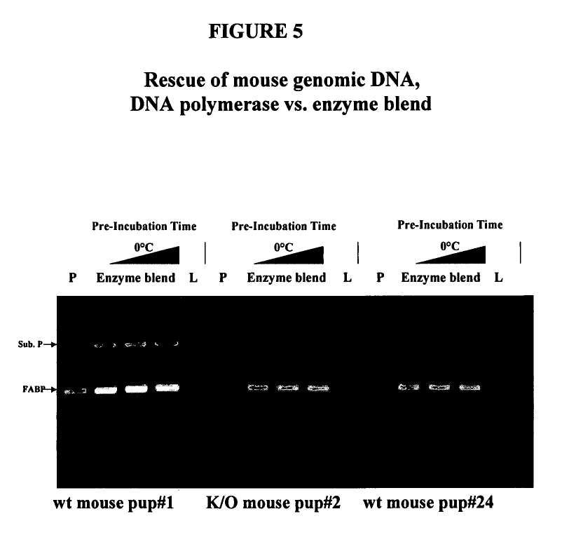

This example demonstrates that the enzymatic components of the Enzyme Blend coexisted without losing functionality. The enzymatic components are AP endonuclease VI and AccuTaq™ LA DNA polymerase. A 1 mM DTT was diluted 1:100 using Taq dilution buffer. The diluted DTT was then added to the Enzyme Blend in the amount of about 0.075 μl/rxn. The Enzyme Blend has about 0.25 μl of AP endonuclease VI (or 25 Units) and about 0.5 μl of AccuTaq™ LA DNA polymerase. A 1 ul amount of the Enzyme Blend was added to damaged DNA and the mixture was incubated on ice for 1 to 5 minutes. To confirm that the Enzyme Blend was able to repair the damaged DNA, a sequence analysis was performed. Sequencing analysis shows over 98% recovery of correct original base sequence after treatment with the AP endonuclease VI/AccuTaq™ blend as compared to the damaged template, which was too damaged to sequence. FIG. 3 shows the results of this experiment.

### Example 5

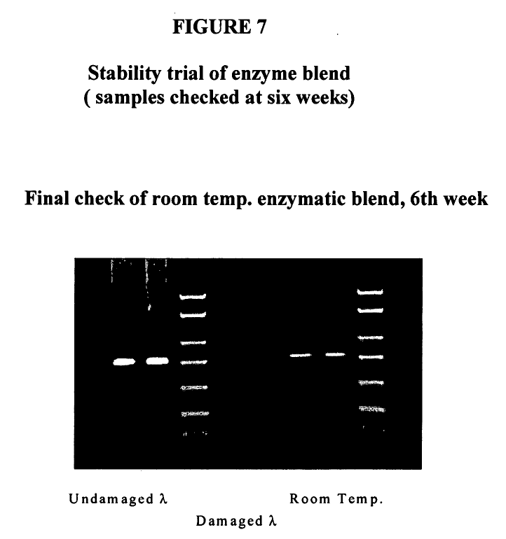

This example demonstrates that the blend was stable and retained its function after storage at room temperature (about 25° C.) or 37° C. for at least one month. The DNA template (742 bp) was damaged with formic acid treatment for 7.5 minutes. FIGS. 6 and 7 show that the stability of the Enzyme Blend was maintained after its storage at room temperature or 37° C. for at least a month.

### Example 6

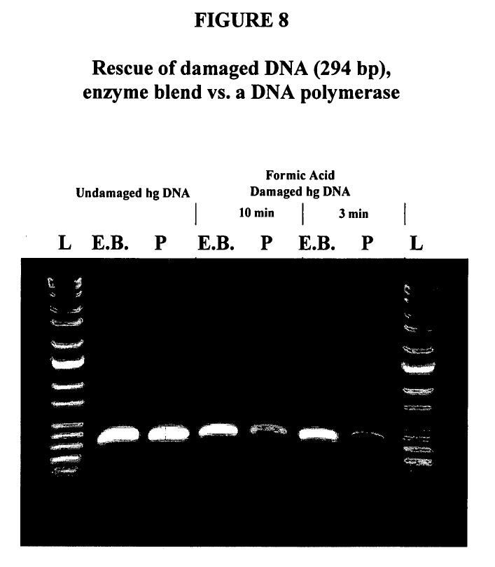

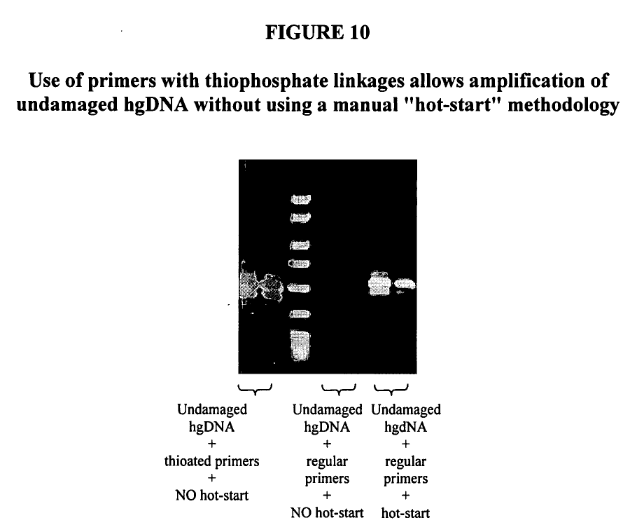

This experiment demonstrates that the Enzyme Blend repaired short human genomic DNA (“gDNA”). The Enzyme Blend was added to 200 ng of a 527 bp human genomic DNA template. The DNA was intentionally damaged by formic acid treatment. For amplification, a manual inactivation or “hotstart” step was performed. A varying amount (2 μM, 0.2 μM, and 0.5 μM) of primers was used in the PCR. FIG. 8 shows the results of the experiment. FIG. 10 shows the results using 527 bp hgDNA, and FIG. 9 shows the results using a 294 bp DNA.

### Example 7

This experiment shows that the Enzyme Blend rescued damaged DNA using varying amounts of AP endonuclease VI over a time-course. Master mixes containing 0, 5, and 50 Units of AP endonuclease VI were added at time points of 0 min., 30 min., 1 hr., 2 hrs., and 3 hrs., using a heat inactivation step to inactivate the AP endonuclease DNA repair enzyme in the Enzyme Blend. When using a large amount of AP endonuclease VI (50 Units), good results could be obtained using a short incubation time (30 min.). Good results could also be obtained when using a small amount of AP endonuclease VI (5 Units) along with a long incubation time (1 hr.). (Data not shown).

### Example 8

### Thiophosphate Primers

This example demonstrates the use of thiophosphate primers with the Enzyme Blend to rescue human genomic DNA (Roche). Heating at 99° C. for 0.5, 1, 3, and 10 minutes in an ABI 9700 created intentionally damaged templates. The ability to amplify either a 5 or 20 kb region of the human-globin gene was tested using oligonucleotides containing thiophosphate (*) linkages (Sigma-Genosys). For amplification of the 5 kb region, oligonucleotide primers HuG5F (21mer, 5′-CCTCAGCCTCAGAATTTG*GC*A-3′) and HuG5R (22mer, 5′-TCTCCCCAACCTCCCCCAT*CT*A-3′) were used. For amplification of the 20 kb amplification, HuG5F, as an anchor primer, and HuG20R (21mer, 5′-TGTTACTTCTCCCCTTCC*TA*T-3′), as a complementary primer, were used.

The PCR reactions were prepared on ice and the reagents were to a final volume of 50 ul. All reactions were performed using an ABI 9700 thermal cycler. Each reaction was stopped with the addition of 10 ul Gel loading solution (Sigma Prod. # G2526) and kept on ice. FIG. 10 shows the results.

### Example 9

### Thiophosphate Primers without AP Endonuclease

The present inventors contemplate that amplification of DNA with proofreading DNA polymerase can be dramatically improved by using oligonucleotide primers having thiophosphate linkages, as described in the previous example. A mixture is formed by adding undamaged DNA, an effective amount of a DNA polymerase, deoxynucleoside 5′ triphosphates, and a pair of oligonucleotide primers having the thiophosphate linkages into a vessel. The pair of primers is sufficiently complementary to predetermined segments of the DNA template, as described in Example 1. The mixture is incubated at, for example, 94° C. for 5 sec. to denature the DNA and then subject to amplification either by PCR as described in the previous examples (or with slightly modified conditions as needed to obtain optimum or desired results) or by rolling circle amplification. The present inventors anticipate that the amplification will result in increased yield of products as compared to amplification with unmodified oligonucleotide primers (that is, primers that do not have thiophosphate linkages).

### Example 10

### Endonuclease IV

This example demonstrates the amplification of DNA using Endonuclease IV (Endo IV) with AccuTaq™ LA DNA polymerase and rescue of DNA using Endonuclease IV with the Enzyme Blend. A 5 kb DNA was amplified using the following components: 1× Accutaq LA Buffer (Sigma Prod. # B0194), 400M of dNTP mix (Sigma Prod. #D7295), 200 nM HuG5F & HuG5R primers, either 0.1 ng/ul or 1 ng/ul hgDNA template, 0.05 U/ul of either Accutaq LA (Sigma Prod. #D5553) or the Enzyme Blend, and with or without 0.01 U/ul E. coli Endonuclease IV (Trevigen). Amplification reactions were performed using an ABI 9700 with the following cycling conditions: 1) 94° C. for 30 sec.; 2) 30 cycles of 94° C. for 10 sec, 65° C. for 20 sec, 68° C. for 5 min; and 3) a final extension of 68° C. for 7 min. FIG. 11 shows the results.

### Example 11

### Glycosylase

This example demonstrates the amplification and rescue of DNA using Uracil DNA Glycosylase (UNG) with AccuTaq™ LA DNA polymerase or with the Enzyme Blend, respectively. FIG. 12 shows the results. DNA (5 kb) was amplified using the following components: 1× AccuTaq™ LA Buffer, 400M of dNTP mix, 300 nM HuG5F & HuG5R primers, either 0.1 ng/ul or 1 ng/ul hgDNA template, 0.05 U/ul of either AccuTaq™ LA or the Enzyme Blend, and with or without 0.005 U/ul UNG (Sigma Prod. #U1257). Amplification reactions were performed using an ABI 9700 with the following cycling conditions: 1) 94° C. for 30 sec; 2) 30 cycles of 94° C. for 20 sec, 65° C. for 20 sec, 68° C. for 7 min; and 3) a final extension of 68° C. for 7 min.

For amplification of DNA (20 kb), the following conditions were used: 1× AccuTaq™ LA Buffer, 500M of dNTP mix, 400 nM HuG5F & HuG20R primers, 2% DMSO (Sigma Prod. # D8418), either 1, 2, or 4 ng/ul hgDNA template, 0.05 U/ul of either AccuTaq™ LA DNA polymerase or the Enzyme Blend, and with or without 0.005 U/ul UNG. The amplification reactions were performed using an ABI 9700 with the following cycling conditions: First cycle program: 1) 94° C. for 1 min.; 2) 15 cycles of 94° C. for 20 sec., 63° C. for 30 sec., 68° C. for 20 min. Second cycle program: 1) 15 cycles of 94° C. for 20 sec., 63° C. for 30 sec., 68° C. for 20 min. plus 15 sec. of autoextension per cycle. The autoextension incrementally adds an additional 15 seconds per cycle. Final extension: 68° C. for 10 min.

### Example 12

### Ligase

The present inventors contemplate that the amplification of DNA that is damaged, undamaged, or suspected of being damaged can be improved by the addition of DNA ligase. A mixture containing the DNA, an effective amount of the Enzyme Blend, T4 DNA Ligase, deoxynucleotide 5′ triphosphates and buffer is incubated at 0-50° C. for 0-3 hours. The DNA may be damaged by acid, heat, oxidation or reaction with organic chemicals. Shorter amounts of incubation time are required for templates with light damage, whereas heavily damaged DNA may require longer incubation times. After incubation with the Enzyme Blend and T4 DNA ligase, the reaction is incubated at a temperature and for a time sufficient to inactive the AP endonuclease activity in the Enzyme Blend. This inactivation step is not necessary if the primers have been modified to resist the degradation by the AP endonuclease (e.g. the primers are modified to contain thiophospate linkages). Following the inactivation step, the mixture is subjected to PCR, which comprises the steps of denaturation, annealing and extension, which are then repeated numerous times. After this PCR step, electrophoresis and ethidium bromide staining on a gel may be used to analyze the product. The inventors anticipate that the addition of T4 DNA ligase to the Enzyme Blend to amplify the DNA will result in a higher yield of PCR product with increased specificity when compared to the amplification of damaged DNA without T4 DNA ligase. If a thermostable T4 DNA Ligase, then a pre-PCR incubation step would not be necessary. Higher yield and specificity of the PCR amplicon would also be expected with the thermostable T4 Ligase as compared to an amplification reaction without the thermostable T4 DNA Ligase.

For example, the rescue of damaged DNA can be improved by adding 2 units of thermolabile T4 DNA ligase to a mixture as described in any of the previous examples for rescue. The mixture is subject to a pre-PCR incubation and then subject to PCR.

### Example 13

### Photolyase

The present inventors contemplate that the amplification of DNA that is damaged, undamaged, or suspected of being damaged can be improved by the addition of DNA photolyase. The amplification of the DNA can be improved by adding, for example, 2 units of Thermus thermophilus photolyase to a mixture as described in any of the previous examples for rescue. The mixture is subject to a pre-PCR incubation and then subject to PCR. Specifically, a mixture containing the DNA, an effective amount of the Enzyme Blend, Thermus thermophilus photolyase, deoxynucleotide 5′ triphosphates, and buffer is incubated at about 0-50° C. for about 0-3 hours. If damaged, the DNA may be damaged by acid, heat, UV light, oxidation or reaction with organic chemicals. Shorter amounts of incubation time are required for templates with light damage, whereas heavily damaged DNA may require longer incubation times. After incubation with the Enzyme Blend and Thermus thermophilus photolyase, the reaction is incubated at a temperature and for a time sufficient to inactive the AP endonuclease activity in the Enzyme Blend. This inactivation step is not necessary if the primers have been modified to resist the degradation by the AP endonuclease (e.g the primers are modified to contain thiophospate linkages). Following the inactivation step, the mixture is subjected to PCR, which comprises the steps of denaturation, annealing and extension, which are then repeated numerous times. After this PCR step, the products may be analyzed by loading them onto a gel and subjecting them to electrophoresis. The products in the gel are stained with ethidium bromide. The inventors anticipate that the addition of the Thermus thermophilus photolyase to the Enzyme Blend to amplify the DNA will result in a higher yield of PCR product with increased specificity when compared to the amplification of DNA without the Thermus thermophilus photolyase. Higher yield and specificity of the PCR amplicon would also be expected with a thermostable Thermus thermophilus photolyase as compared to a rescue reaction without the thermostable Thermus thermophilus photolyase.

The examples provided above are for illustrative purposes only, and not to limit the scope of the present invention. In light of the present disclosure, numerous embodiments within the scope of the claims will be apparent to those of ordinary skill in the art.

### REFERENCES

1. Barnes, W. M., PCR amplification of up to 35-kb DNA with highfidelity and high yield from λ bacteriophage templates, Proc. Natl. Acad. Sci. USA, 91:2216-2220 (1994).
2. Cheng, S., et al., Effective amplification of long targets from cloned inserts and human genomic DNA, Proc. Natl. Acad. Sci. USA, 91:5695-5699 (1994).
3. Don, R. H., et al. “Touchdown” PCR to circumvent spurious priming during gene amplification, Nucleic Acids Res., 19:4008 (1991).
4. PCR Technology: Current Innovations, Griffin, H. G., and Griffin, A. M., (Eds.) (CRC Press, Boca Raton, Fla., 1994).
5. PCR Strategies, Innis, M. A., et al. (Eds.) (Academic Press, New York, 1995).
6. PCR Protocols: A Guide to Methods and Applications, Innis, M., et al. (Eds.) (Academic Press, San Diego, Calif., 1990).
7. Lowe, T., et al., A computer program for selection of oligonucleotide primers for polymerase chain reaction, Nucleic Acids Res., 18:1757-1761 (1990).
8. PCR: Essential Data, Newton, C. R., (Ed.) (John Wiley & Sons, New York, 1995).
9. Roux, K. H. Optimization and troubleshooting in PCR, PCR Methods Appl., 4:5185-5194 (1995).
10. Molecular Cloning: A Laboratory Manual, Third Edition, Sambrook, J., et al., (Cold Spring Harbor Laboratory Press, New York, 2000).
11. Rychlik and Rhoads, Nucl. Acids Res., 17:8543-8551 (1989).
12. Di Bernardo, G., S. Del Gaudio, M. Cammarota, U. Galderisi, A. Cascino, and M. Cipollaro, Enzymatic repair of selected cross-linked homoduplex molecules enhances nuclear gene rescue from Pompeii and Herculaneum remains, Nucleic Acids Res., 30:06 (2002).
13. Greagg, M. A., M. J. Fogg, G. Panayotou, S. J. Evans, B. A. Connolly, and L. H. Pearl, A read-a head function in archaeal DNA polymerases detects promutagenic template-strand uracil, PNAS, 96:9045-9050 (1999).
14. Hofreiter, M., V. Jaenicke, D. Serre, A. Haeseler Av, and S. Paabo, DNA sequences from multiple amplifications reveal artifacts induced by cytosine deamination in ancient DNA, Nucleic Acids Res, 29:4793-9 (2001).
15. Hofreiter, M., D. Serre, H. N. Poinar, M. Kuch, and S. Paabo, Ancient DNA, Nat Rev Genet, 2:353-9 (2001).
16. Mol, C. D., D. J. Hosfield, and J. A. Tainer, Abasic site recognition by two apurinic/apyrimidinic endonuclease families in DNA base excision repair: the 3′ ends justify the means, Mutat Res, 460:211-29 (2000).
17. Paabo, S., Ancient DNA: extraction, characterization, molecular cloning, and enzymatic amplification, Proc Natl Acad Sci USA, 86:1939-43 (1989).
18. Shida, T., M. Noda, and J. Sekiguchi, Cleavage of single-and double-stranded DNAs containing an abasic residue by Escherichia coli AP endonuclease VI (AP endonuclease VI), Nucl. Acids. Res., 24:4572-4576 (1996).

1. An Enzyme Blend for use with DNA comprising a DNA polymerase and a means for repairing an apurinic/apyrimidinic (AP) damage in DNA.

2. The Enzyme Blend of claim 1, wherein the means for repairing an AP damage in DNA is an AP endonuclease DNA repair enzyme.

3. The Enzyme Blend of claim 1, wherein the DNA has been damaged, is suspected of being damaged, or is undamaged.

4. The Enzyme Blend of claim 2, wherein the AP endonuclease DNA repair enzyme is AP endonuclease VI, REF1, APEX, Endonuclease IV, APNI, APE1 (human endonuclease 1), or FEN-1.

5. The Enzyme Blend of claim 4, wherein the AP endonuclease DNA repair enzyme is AP endonuclease VI.

6. The Enzyme Blend of claim 1, further comprising a stabilizing agent.

7. The Enzyme Blend of claim 6, wherein the stabilizing agent is 1,4-dithioerythritol, DL-dithiothreitol, 2-mercaptoethanol, 2-mercaptoethanolamine, fericyanide, hydrazine, borane, or phosphine.

8. The Enzyme Blend of claim 1, further comprising a ligase.

9. The Enzyme Blend of claim 8, wherein the ligase is T4 DNA ligase.

10. The Enzyme Blend of claim 1, further comprising a DNA glycosylase.

11. The Enzyme Blend of claim 10, wherein the DNA glycosylase is uracil N-glycosylase.

12. The Enzyme Blend of claim 1, further comprising endonuclease IV.

13. The Enzyme Blend of claim 1, further comprising DMSO.

14. The Enzyme Blend of claim 1, further comprising a photolyase.

15. The Enzyme Blend of claim 14, wherein the photolyase is Thermus thermophilus photolyase.

16. The Enzyme Blend of claim 5 comprising:
a) 0.1-25 units/ul DNA polymerase; and
b) 5-50 units/ul AP endonuclease VI.

17. The Enzyme Blend of claim 16, further comprising:
a) 1-15 mM DTT; and
b) 10-50% v/v glycerol.

18. An Enzyme Blend comprising:
a) 2.5 units/ul DNA polymerase;
b) 5-50 units/ul AP endonuclease VI;
c) 10 mM Tris-HCl pH 8.0;
d) 150 mM KCl;
e) 100 ug/ml BSA;
f) 0.075 mM EDTA;
g) 7.5 mM DTT;
h) 0.25% v/v Tween 20;
i) 0.25% v/v IGEPAL CA-630; and
j) 50% v/v glycerol.

19. A kit comprising the Enzyme Blend of claim 1.

20. The kit of claim 19, wherein the means for repairing an AP damage in DNA is an AP endonuclease DNA repair enzyme.

21. The kit of claim 20, wherein the AP endonuclease DNA repair enzyme in the Enzyme Blend is AP endonuclease VI, REF1, APEX, Endonuclease IV, APNI, APE1 (human endonuclease 1), or FEN-1.

22. The kit of claim 21, wherein the AP endonuclease DNA repair enzyme is AP endonuclease VI.

23. The kit of claim 22, wherein the Enzyme Blend comprises:
a) 0.1-25 units/ul DNA polymerase; and
b) 5-50 units/ul AP endonuclease VI.

24. The kit of claim 23, wherein the Enzyme Blend further comprises:
a) 1-15 mM DTT; and
b) 10-50% v/v glycerol.

25. A kit comprising the Enzyme Blend of claim 18.

26. A method for repairing DNA that is damaged or suspected of being damaged, comprising:
a) forming a mixture comprising the DNA, an effective amount of the Enzyme Blend of claim 1, and deoxynucleoside 5′ triphosphates; and
b) incubating the mixture at 0° C.-99° C. from about 0 sec. to about 3 hrs.

27. The method of claim 26, wherein the mixture is incubated for 0° C.-50° C. from about 0 sec. to about 1 hr. in step (b).

28. The method of claim 26, wherein the DNA has a size of at least about 200 base pairs.

29. The method of claim 28, wherein the DNA has a size of at least about 500 base pairs.

30. The method of claim 26, wherein the DNA has a size of less than about 22,000 base pairs.

31. The method of claim 30, wherein the DNA has a size of less than about 1,000 base pairs.

32. The method of claim 26, wherein the DNA has a size of about 50 base pairs to about 500 base pairs.

33. The method of claim 26, wherein the DNA has a size of about 15,500 base pairs to about 22,000 base pairs.

34. The method of claim 26, wherein the means for repairing an AP damage in DNA is an AP endonuclease DNA repair enzyme.

35. A method for amplification of DNA that is damaged, undamaged, or suspected of being damaged, comprising:
a) forming a mixture comprising the DNA, an effective amount of the Enzyme Blend of claim 1, deoxynucleoside 5′ triphosphates, and a pair of oligonucleotide primers, wherein the pair of primers is substantially complementary to segments of the DNA;
b) preincubating the mixture at 0° C.-99° C. from about 0 sec. to about 3 hrs.;
c) denaturing the DNA; and
d) amplifying the DNA.

36. The method of claim 35, wherein the mixture is incubated at 0° C.-50° C. from about 0 sec. to about 1 hr. in step (b).

37. The method of claim 35, wherein step (d) is a polymerase chain reaction that comprises the steps of denaturation, annealing, and extension.

38. The method of claim 35, wherein step (d) is a rolling circle amplification.

39. The method of claim 35, wherein the pair of oligonucleotide primers have thiophosphate linkages.

40. The method of claim 39, wherein the thiophosphate linkages are located on the last two nucleotides at the 3′ end of each oligonucleotide primer.

41. The method of claim 35, wherein the means for repairing an AP damage in DNA is an AP endonuclease DNA repair enzyme.

42. The method of claim 41, wherein the AP endonuclease DNA repair enzyme in the Enzyme Blend is AP endonuclease VI, REF1, APEX, Endonuclease IV, APNI, APE1 (human endonuclease 1), or FEN-1.

43. The method of claim 42, wherein the AP endonuclease DNA repair enzyme is AP endonuclease VI.

44. The method of claim 43, wherein the Enzyme Blend comprises:
a) 0.1-25 units/ul DNA polymerase; and
b) 5-50 units/ul AP endonuclease VI.

45. The method of 44, wherein the Enzyme Blend further comprises:
a) 3-15 mM DTT; and
b) 16-50% v/v glycerol.

46. The method of claim 35, where any or all of the steps are automated.

47. The method of claim 35, wherein the DNA has a size of at least about 200 base pairs.

48. The method of claim 47, wherein the DNA has a size of at least 500 base pairs.

49. The method of claim 35, wherein the DNA has a size of less than about 22,000 base pairs.

50. The method of claim 49, wherein the DNA has a size of less than about 1,000 base pairs.

51. The method of claim 35, wherein the damaged DNA has a size of about 50 base pairs to about 500 base pairs.

52. The method of claim 35, wherein the DNA has a size of about 15,500 base pairs to about 22,000 base pairs.

53. A method for amplification of DNA that is damaged, undamaged, or suspected of being damaged, comprising:
a) forming a mixture comprising the DNA, an effective amount of the Enzyme Blend of claim 18, deoxynucleoside 5′ triphosphates, and a pair of oligonucleotide primers, wherein the pair of primers is substantially complementary to segments of the DNA;
b) preincubating the mixture at 0° C.-99° C. from about 0 sec. to about 3 hrs.;
c) denaturing the DNA; and
d) amplifying the DNA.

54. The method of claim 53, wherein the mixture is incubated at 0° C.-50° C. from about 0 sec. to about 1 hr. in step (b).

55. A method for amplification of DNA that is damaged, undamaged, or suspected of being damaged, comprising:
a) forming a mixture comprising the DNA, an effective amount of the Enzyme Blend of claim 1, and deoxynucleoside 5′ triphosphates;
b) preincubating the mixture at a temperature of 0° C.-99° C. from about 0 sec. to about 3 hrs.;
c) denaturing the DNA;
d) incubating the mixture at a temperature sufficient to inactivate an AP endonuclease DNA repair enzyme in the Enzyme Blend and for a duration of time necessary to add a pair of oligonucleotide primers to the mixture, wherein the pair of primers is substantially complementary to segments of the DNA;
e) adding the pair of oligonucleotide primers to the mixture; and
f) amplifying the DNA.

56. The method of claim 55, wherein the mixture is incubated at 0° C.-50° C. from about 0 sec. to about 1 hr. in step (b).

57. The method of claim 55, wherein step (f) is a polymerase chain reaction that comprises the steps of denaturation, annealing, and extension.

58. The method of claim 55, wherein step (f) is a rolling circle amplification.

59. The method of claim 55, wherein the means for repairing an AP damage in DNA is an AP endonuclease DNA repair enzyme.

60. The method of claim 59, wherein the AP endonuclease DNA repair enzyme in the Enzyme Blend is AP endonuclease VI, REF1, APEX, Endonuclease IV, APNI, APE1 (human endonuclease 1), or FEN-1.

61. The method of claim 60, wherein the AP endonuclease DNA repair enzyme is AP endonuclease VI.

62. The method of claim 61, wherein the Enzyme Blend comprises:
a) 0.1-25 units/ul DNA polymerase; and
b) 5-50 units/ul AP endonuclease VI.

63. The method of claim 62, wherein the Enzyme Blend further comprises:
a) 3-15 mM DTT; and
b) 16-50% v/v glycerol.

64. The method of claim 55, wherein any or all of the steps are automated.

65. The method of claim 55, wherein the DNA has a size of at least about 200 base pairs.

66. The method of claim 65, wherein the DNA has a size of at least 500 base pairs.

67. The method of claim 55, wherein the DNA has a size of less than 22,000 base pairs.

68. The method of claim 67, wherein the DNA has a size of less than about 1,000 base pairs.

69. The method of claim 55, wherein the DNA has a size of about 50 base pairs to about 500 base pairs.

70. The method of claim 55, wherein the DNA has a size of about 15,500 base pairs to about 22,000 base pairs.

71. A method for amplification of DNA that is damaged, undamaged, or suspected of being damaged, comprising:
a) forming a mixture comprising the DNA, an effective amount of the Enzyme Blend of claim 18, and deoxynucleoside 5′ triphosphates;
b) preincubating the mixture at a temperature of 0° C.-99° C. from about 0 sec. to about 3 hrs.;
c) denaturing the DNA;
d) incubating the mixture at a temperature sufficient to inactivate an AP endonuclease DNA repair enzyme in the Enzyme Blend and for a duration of time necessary to add a pair of oligonucleotide primers to the mixture, wherein the pair of primers is substantially complementary to segments of the DNA;
e) adding the pair of oligonucleotide primers to the mixture; and
f) amplifying the DNA.

72. The method of claim 71, wherein the mixture is incubated at 0° C.-50° C. from about 0 sec. to about 1 hr. in step (b).

73. A method for preparation of an Enzyme Blend comprising combining a DNA polymerase with a means for repairing an AP damage in DNA in a vessel to form a blend.

74. The method of claim 73, wherein the means for repairing an AP damage in DNA is an AP endonuclease DNA repair enzyme.

75. The method of claim 74, wherein the AP endonuclease DNA repair enzyme is AP endonuclease VI, REF1, APEX, Endonuclease IV, APNI, APE1 (human endonuclease 1), or FEN-1.

76. The method of claim 75, wherein the AP endonuclease DNA repair enzyme is AP endonuclease VI.

77. The method of claim 71, wherein the Enzyme Blend further comprises a stabilizing agent.

78. The method of claim 77, wherein the stabilizing agent is 1,4-dithioerythritol, DL-dithiothreitol, 2-mercaptoethanol, 2-mercaptoethanolamine, fericyanide, hydrazine, borane, or phosphine.

79. The method of claim 71, wherein the Enzyme Blend further comprises a ligase.

80. The method of claim 79, wherein the ligase is T4 DNA ligase.

81. The method of claim 71, wherein the Enzyme Blend further comprises a DNA glycosylase.

82. The method of claim 81, wherein the DNA glycosylase is Uracil N-glycosylase.

83. The method of claim 71, wherein the Enzyme Blend further comprises endonuclease IV.

84. The method of claim 71, wherein the Enzyme Blend further comprises DMSO.

85. The method of claim 71, wherein the Enzyme Blend further comprises a photolyase.

86. The method of claim 85, wherein the photolyase is Thermus thermophilus photolyase.

87. A method for amplification of DNA that is damaged or suspected of being damaged comprising:
a) forming a mixture comprising the DNA, an effective amount of DNA polymerase, an effective amount of a means for repairing an AP damage in DNA, deoxynucleoside 5′ triphosphates, and a pair of oligonucleotide primers, wherein the pair of primers is substantially complementary to segments of the DNA;
b) preincubating the mixture at 0° C.-99° C. from about 0 sec. to about 3 hrs.;
c) denaturing the DNA; and
d) amplifying the DNA,
wherein the DNA has a size from about 50 base pairs to about 500 base pairs or has a size from about 15,500 base pairs to about 22,000 base pairs.

88. The method of claim 87, wherein the mixture is incubated at 0° C.-50° C. from about 0 sec. to about 1 hr. in step (b).

89. The method of claim 87, wherein the means for repairing an AP damage in DNA is an AP endonuclease DNA repair enzyme.

90. A method for rescue of DNA that is damaged or suspected of being damaged comprising:
a) forming a mixture comprising the DNA, an effective amount of DNA polymerase, an effective amount of a means for repairing an AP damage in DNA, and deoxynucleoside 5′ triphosphates;
b) preincubating the mixture at a temperature of 0° C.-99° C. from about 0 sec. to about 3 hrs.;
c) denaturing the DNA;
d) incubating of the mixture at a temperature sufficient to inactivate the AP endonuclease DNA repair enzyme and for a duration of time necessary to add a pair of oligonucleotide primers to the mixture, wherein the pair of primers is substantially complementary to segments of the DNA;
e) adding the pair of oligonucleotide primers to the mixture; and
f) amplifying the DNA, wherein the DNA has a size from about 50 base pairs to about 500 base pairs or has a size from about 15,500 base pairs to about 22,000 base pairs.

91. The method of claim 90, wherein the mixture is incubated at 0° C.-50° C. from about 0 sec. to about 1 hr. in step (b).

92. The method of claim 90, wherein the means for repairing an AP damage in DNA is an AP endonuclease DNA repair enzyme.

93. An improved method for amplification of undamaged DNA comprising:
a) forming a mixture comprising the DNA, an effective amount of a DNA polymerase, deoxynucleoside 5′ triphosphates, and a pair of oligonucleotide primers having thiophosphate linkages, wherein the pair of primers is substantially complementary to segments of the DNA;
b) denaturing the DNA; and
c) amplifying the DNA.

94. The method of claim 93, wherein step (c) is a polymerase chain reaction that comprises the steps of denaturation, annealing, and extension.

95. The method of claim 93, wherein step (c) is a rolling circle amplification.

96. The method of claim 93, wherein the thiophosphate linkages are located on the last two nucleotides at the 3′ end of each oligonucleotide primer.
